SEQUENCE LISTING (37 sequences)

SEQ ID NO: 1 - protein, 7 aa, Human immunodeficiency virus
KRRQRRR

SEQ ID NO: 2 - protein, 16 aa, Artificial Sequence
  Description of Artificial Sequence Synthetic peptide
RQIKIWFQNRRMKWKK

SEQ ID NO: 3 - protein, 34 aa, Human herpesvirus
DAATATRGRSAASRPTERPRAPARSASRPRRPVD

SEQ ID NO: 4 - protein, 16 aa, Artificial Sequence
  Description of Artificial Sequence Synthetic peptide
AAVALLPAVLLALLAP

SEQ ID NO: 5 - protein, 16 aa, Artificial Sequence
  Description of Artificial Sequence Synthetic peptide
AAVLLPVLLPVLLAAP

SEQ ID NO: 6 - protein, 15 aa, Homo sapiens
VTVLALGALAGVGVG

SEQ ID NO: 7 - protein, 17 aa, Human immunodeficiency virus
GALFLGWLGAAGSTMGA

SEQ ID NO: 8 - protein, 17 aa, Caiman crocodilus
MGLGLHLLVLAAALQGA

SEQ ID NO: 9 - protein, 24 aa, Homo sapiens
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Influenza virus
GLFGAIAGFIENGWEG

SEQ ID NO: 15 - protein, 16 aa, Human parainfluenza virus
FFGAVIGTIALGVATA

SEQ ID NO: 16 - protein, 16 aa, Respiratory Syncytial Virus
FLGFLLGVGSAIASGV

SEQ ID NO: 17 - protein, 16 aa, Human immunodeficiency virus
GVFVLGFLGFLATAGS

SEQ ID NO: 18 - protein, 16 aa, Ebola virus
GAAIGLAWIPYFGPAA

SEQ ID NO: 19 - protein, 56 aa, Artificial Sequence
  Description of Artificial Sequence Synthetic polypeptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic polypeptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic polypeptide
ACTCPNCKEGGGRGTNLGKKKQHICHIPGCGKVYGKTSHLRAHLRWHSGERPFVC

SEQ ID NO: 22 - protein, 56 aa, Artificial Sequence
  Description of Artificial Sequence Synthetic polypeptide
ACSCPNCREGEGRGSNEPGKKKQHICHIEGCGKVYGKTSHLRAHLRWHTGERPFIC

SEQ ID NO: 23 - protein, 60 aa, Drosophila melanogaster
RCTCPNCTNEMSGLPPIVGPDERGRKQHICHIPGCERLYGKASHLKTHLRWHTGERPFLC

SEQ ID NO: 24 - protein, 58 aa, Drosophila melanogaster
TCDCPNCQEAERLGPAGVHLRKKNIHSCHIPGCGKVYGKTSHLKAHLRWHTGERPFVC

SEQ ID NO: 25 - protein, 53 aa, Caenorhabditis elegans
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic polypeptide
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic peptide
WWETWKPFQCRICMRNFSTRQARRNHRRRHR

SEQ ID NO: 28 - protein, 16 aa, Artificial Sequence
  Description of Artificial Sequence Synthetic peptide
GKINLKALAALAKKIL

SEQ ID NO: 29 - protein, 16 aa, Artificial Sequence
  Description of Artificial Sequence Synthetic peptide
RVIRVWFQNKRCKDKK

SEQ ID NO: 30 - protein, 39 aa, Artificial Sequence
  Description of Artificial Sequence Synthetic peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic peptide
GEQIAQLIAGYIDIILKKKKSK

SEQ ID NO: 32 - protein, 78 aa, Homo sapiens
FPIPLPYCWLCRALIKRIQAMIPKGALRVAVAQVCRVVPLVAGGICQCLAERYSVILLDTLLGRMLPQLVCRLVLRCS

SEQ ID NO: 33 - DNA, 21 bp, Artificial Sequence
  Description of Artificial Sequence Synthetic oligonucleotide
gactacacaaatcagcgattt

SEQ ID NO: 34 - RNA, 19 nt, Artificial Sequence
  Description of Artificial Sequence Synthetic oligonucleotide
uucuccgaacgugucacgu

SEQ ID NO: 35 - protein, 36 aa, Artificial Sequence
  Description of Artificial Sequence Synthetic peptide
KGSKKAVTKAQKKDGKKRKRSRKESYSVYVYKVLKQ

SEQ ID NO: 36 - protein, 28 aa, Artificial Sequence
  Description of Artificial Sequence Synthetic peptide
KETWWETWWTEWSQPGRKKRRQRRRPPQ

SEQ ID NO: 37 - protein, 23 aa, Artificial Sequence
  Description of Artificial Sequence Synthetic peptide
CXXXXCXXXXXXXXXXXXHXXXH

(19) United States
(12) Patent Application Publication
(10) Pub. No.: US 2007/0281900 A1
(43) Pub. Date: Dec. 6, 2007

(54) COMPOSITIONS AND METHODS FOR LIPID AND POLYPEPTIDE BASED siRNA INTRACELLULAR DELIVERY

(75) Inventors: Kunyuan Cui, Bothell, WA (US); Dong Liang, Bothell, WA (US); David S. Sweedler, Louisville, CO (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/743,480

(22) Filed: May 2, 2007

Related U.S. Application Data
(60) Provisional application No. 60/798,243, filed on May 5, 2006.

(51) Int. Cl. A61K 48/00; C12N 15/88

(52) U.S. Cl. 514/44; 435/458

(57) ABSTRACT

A composition of an interfering RNA comprising a double-stranded RNA (dsRNA) molecule having a double-stranded region of from about 15 to about 40 base pairs, a peptide having a hydrophobic region and a cationic region, and a non-cationic phospholipid, and uses thereof.

This application claims the benefit under 35 U.S.C. § 119(e) of U.S. Provisional Application No. 60/798,243, filed May 5, 2006, which is hereby incorporated by reference in its entirety.

### BACKGROUND OF THE INVENTION

Delivering nucleic acids into animal and plant cells to achieve a specific biological effect has long been an important object of molecular biology research and development. Recent developments in the areas of gene therapy, antisense therapy and RNA interference (RNAi) therapy have created a need to develop more efficient means for introducing nucleic acids into cells.

RNA interference is a process of sequence-specific post transcriptional gene silencing in cells initiated by a double-stranded (ds) polynucleotide, usually a dsRNA, that is homologous in sequence to a portion of a targeted messenger RNA (mRNA). Introduction of a suitable dsRNA into cells leads to destruction of endogenous, cognate mRNAs (i.e., mRNAs that share substantial sequence identity with the introduced dsRNA). The dsRNA molecules are cleaved by an RNase III family nuclease called dicer into short-interfering RNAs (siRNAs), which are 19-23 nucleotides (nt) in length. The siRNAs are then incorporated into a multicomponent nuclease complex known as the RNA-induced silencing complex or “RISC.” The RISC identifies mRNA substrates through their homology to the siRNA, and effectuates silencing of gene expression by binding to and destroying the targeted mRNA.

RNA interference is emerging a promising technology for modifying expression of specific genes in plant and animal cells, and is therefore expected to provide useful tools to treat a wide range of diseases and disorders amenable to treatment by modification of endogenous gene expression.

A variety of methods are available for delivering nucleic acids artificially into cells. These include transfection via calcium phosphate, cationic lipid, and lipsomal delivery. Nucleic acids can also be introduced into cells by electroporation and viral transduction. However, there are disadvantages to these methods. With viral gene delivery, there is a possibility that the replication deficient virus used as a delivery vehicle may revert to wild-type thus becoming pathogenic. Electroporation suffers from poor gene-transfer efficiency and therefore has limited clinical application. Finally, transfection of cells by cationic lipids may also be limited by poor efficiency and high cytotoxicity.

Thus, there remains a long-standing need in the art for better tools and methods to deliver nucleic acids, peptides and other pharmacological agents into cells, particularly in view of the fact that existing techniques for delivering cargo into cells are limited by poor efficiency and/or high toxicity of the delivery reagents. Related needs exist for improved methods and formulations to deliver an effective amount, in an active and enduring state, and using non-toxic delivery vehicles, to selected cells, tissues, or compartments to mediate regulation of gene expression in a manner that will alter a phenotype or disease state of the targeted cells.

### BRIEF SUMMARY OF THE INVENTION

One aspect of the invention is a composition for delivery of a RNA molecule to a cell, comprising: a double stranded RNA (dsRNA) molecule of about 15 to about 40 base pairs; a peptide, comprising a hydrophobic region and a cationic region; and a non-cationic phospholipid. In one embodiment, the non-cationic lipid is selected from the group consisting of a neutral lipid, zwitterionic lipid and an anionic lipid. In another embodiment, the non-cationic phospholipid is selected from the group consisting of 1,2-dioleoyl-sn-glycero-3-phosphoethanolamine (DOPE); diphytanoylphosphatidylethanolamine (DPhPE); cholesterol hemisuccinate salt (CHEMS); cholesterol; 1,2-distearoyl-sn-glycero-3-phosphocholine (DSPC); 1,2-dipalmitoyl-sn-glycero-3-phosphocholine (DPPC) and 1,2-dimyristoylamido-1,2-deoxyphosphatidylcholine (DDPC), 1,2-dipalmitoyl-sn-glycero-3-phosphatidylserine (DPPS), 1,2-distearoyl-sn-glycero-3-phosphatidylserine (DSPS), and phosphatidic acid derived from any one of the above lipids. In another embodiment, the peptide has at least three cationic amino acids in a five amino acid region within ten amino acids from a terminus of the peptide. In a related embodiment, the peptide has at least three hydrophobic amino acids in a five amino acid region within ten amino acids from a terminus of the peptide. In another related embodiment, the hydrophobic amino acids are near to one terminus of the peptide and the cationic amino acids are near the other terminus of the peptide. In a preferred embodiment, the hydrophobic amino acids are separated from the cationic amino acids by at least three amino acids. In a specific embodiment, the amino acid sequence of the peptide is: KETWWETWWTEWSQPGRKKRRQRRRPPQ (SEQ ID NO: 36). In another embodiment, the pharmaceutical composition further comprises a cationic lipid, preferably in which the ratio of non-cationic to cationic lipid is greater than about 1:1 (w:w).

Another aspect of the invention is a composition for inhibiting expression of a target gene in a cell, comprising: a double stranded RNA (dsRNA) molecule of about 15 to about 40 base pairs, having sequence homology to a sequence of the gene; a peptide, comprising a hydrophobic region and a cationic region; and a non-cationic phospholipid. In one embodiment, the non-cationic lipid is selected from the group consisting of a neutral lipid, zwitterionic lipid and an anionic lipid. In another embodiment, the non-cationic phospholipid is selected from the group consisting of 1,2-dioleoyl-sn-glycero-3-phosphoethanolamine (DOPE); diphytanoylphosphatidylethanolamine (DPhPE); cholesterol hemisuccinate salt (CHEMS); cholesterol; 1,2-distearoyl-sn-glycero-3-phosphocholine (DSPC); 1,2-dipalmitoyl-sn-glycero-3-phosphocholine (DPPC) and 1,2-dimyristoylamido-1,2-deoxyphosphatidylcholine (DDPC), 1,2-dipalmitoyl-sn-glycero-3-phosphatidylserine (DPPS), 1,2-distearoyl-sn-glycero-3-phosphatidylserine (DSPS), and phosphatidic acid derived from any one of the above lipids. In another embodiment, the peptide has at least three cationic amino acids in a five amino acid region within ten amino acids from a terminus of the peptide. In a related embodiment, the peptide has at least three hydrophobic amino acids in a five amino acid region within ten amino acids from a terminus of the peptide. In a related embodiment, the hydrophobic amino acids are near to one terminus of the peptide and the cationic amino acids are near the other terminus of the peptide. In a preferred embodiment, the hydrophobic amino acids are separated from the cationic amino acids by at least three amino acids. In a specific embodiment, the amino acid sequence of the peptide is: KETWWETWWTEWSQPGRKKRRQRRRPPQ (SEQ ID NO: 36). In another embodiment, the pharmaceutical composition further comprises a cationic lipid, preferably in which the ratio of non-cationic to cationic lipid is greater than about 1:1 (w:w).

Another aspect of the invention is a method for delivering a RNA molecule to a cell, comprising: preparing a composition comprising: a double stranded RNA (dsRNA) molecule of about 15 to about 40 base pairs; a peptide, comprising a hydrophobic region and a cationic region; and a non-cationic phospholipid; and treating a cell with said composition.

Another aspect of the invention is a method for inhibiting expression of a gene in a cell comprising preparing a pharmaceutical composition comprising: a double stranded RNA (dsRNA) molecule of about 15 to about 40 base pairs, having sequence homology to a sequence of the gene; a peptide, comprising a hydrophobic region and a cationic region; and a non-cationic phospholipid; and treating a cell with said pharmaceutical composition.

Another aspect of the invention is a method for inhibiting expression of a gene in a mammal comprising: preparing a pharmaceutical composition comprising a double stranded RNA (dsRNA) molecule of about 15 to about 40 base pairs, having sequence homology to a sequence of the gene; a peptide, comprising a hydrophobic region and a cationic region; and a non-cationic phospholipid; and administering said pharmaceutical composition to said mammal.

### BRIEF DESCRIPTION OF THE DRAWINGS

FIG. 1. FIG. 1 shows the LacZ activity of 9L/LacZ cells as measured by absorbance for non-transfected cells and cells transfected with the lipid groups L1 through L7 at a concentration of 7 nM with either the LacZ or Qneg siRNA. The non-transfected (untrn) cells were used as a base-line for comparison with the LacZ and Qneg siRNA transfections.

FIG. 2. FIG. 2 shows the LacZ activity of 9L/LacZ cells as measured by absorbance for non-transfected cells and cells transfected with the lipid groups L1 through L7 at a concentration of 20 nM with either the LacZ or Qneg siRNA. FIG. 2 also includes cells transfected with HYPERFECT™ (HPF). The non-transfected (untrn) cells were used as a base-line for comparison with the LacZ and Qneg siRNA transfections.

FIG. 3. FIG. 3 shows the LacZ activity of 9L/LacZ cells as measured by absorbance for non-transfected cells and cells transfected with the lipid groups L1 through L7 at a concentration of 60 nM with either the LacZ or Qneg siRNA. The non-transfected (untrn) cells were used as a base-line for comparison with the LacZ and Qneg siRNA transfections.

FIG. 4. FIG. 4 shows the LacZ activity of 9L/LacZ cells as measured by absorbance for non-transfected cells, PN73 (2 μM) alone and either LacZ or Qneg siRNA transfected cells and cells transfected with lipid groups L1 through L7 at a concentration of 7 nM in combination with 2 μM PN73 and either the LacZ or Qneg siRNA. The non-transfected (untrn) cells were used as a base-line for comparison with the LacZ and Qneg siRNA transfections.

FIG. 5. FIG. 5 shows the LacZ activity of 9L/LacZ cells as measured by absorbance for non-transfected cells, PN73 (2 μM) alone and either LacZ or Qneg siRNA transfected cells and cells transfected with lipid groups L1 through L7 at a concentration of 20 nM in combination with 2 μM PN73 and either the LacZ or Qneg siRNA. The non-transfected (untrn) cells were used as a base-line for comparison with the LacZ and Qneg siRNA transfections.

FIG. 6. FIG. 6 shows the LacZ activity of 9L/LacZ cells as measured by absorbance for non-transfected cells, PN73 (2 μM) alone and either LacZ or Qneg siRNA transfected cells and cells transfected with lipid groups L1 through L7 at a concentration of 60 nM in combination with 2 μM PN73 and either the LacZ or Qneg siRNA. The non-transfected (untrn) cells were used as a base-line for comparison with the LacZ and Qneg siRNA transfections.

FIG. 7. FIG. 7 shows the LacZ activity of 9L/LacZ cells as measured by absorbance for non-transfected cells, PN183 (7 μM) alone and either LacZ or Qneg siRNA transfected cells and cells transfected with the lipid groups L1 through L7 at a 7 nM concentration in combination with 7 μM PN183 and either the LacZ or Qneg siRNA. The non-transfected (untrn) cells were used as a base-line for comparison with the LacZ and Qneg siRNA transfections.

FIG. 8. FIG. 8 shows the LacZ activity of 9L/LacZ cells as measured by absorbance for non-transfected cells, PN183 (7 μM) alone and either LacZ or Qneg siRNA transfected cells and cells transfected with the lipid groups L1 through L7 at a 20 nM concentration in combination with 7 μM PN183 and either the LacZ or Qneg siRNA. The non-transfected (untrn) cells were used as a base-line for comparison with the LacZ and Qneg siRNA transfections.

FIG. 9. FIG. 9 shows the LacZ activity of 9L/LacZ cells as measured by absorbance for non-transfected cells, PN183 (7 μM) alone and either LacZ or Qneg siRNA transfected cells and cells transfected with the lipid groups L1 through L7 at a 60 nM concentration in combination with 7 μM PN183 and either the LacZ or Qneg siRNA. The non-transfected (untrn) cells were used as a base-line for comparison with the LacZ and Qneg siRNA transfections.

### DETAILED DESCRIPTION OF THE INVENTION

The present invention satisfies these needs and fulfills additional objects and advantages by providing novel compositions and methods that employ a short interfering nucleic acid (siNA), or a precursor thereof, in combination with a polypeptide and one or more lipids. The polypeptide is a natural or artificial polypeptide. The lipid is a non-cationic lipid or a combination of a non-cationic lipid and a cationic lipid.

A non-cationic lipid includes, but is not limited to neutral lipid, also termed a “helper lipid,” has a zero net charge at the pH where it is considered to be neutral; a zwitterionic lipid, meaning a lipid that has a net zero charge as a result of the presence of both a single positive charge and single negative charge within the lipid separated by more than one atom; and an anionic lipid and an anionic lipid, meaning a lipid that has net anionic charge.

Non-limiting examples of anionic lipids include phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines and modified and derivatives forms thereof.

The combination of the polypeptide with one or more non-cationic lipids or a combination of a non-cationic lipid and a cationic lipid with the siNA enhances the cellular uptake of the siNA and permits siNA mediated reduction of a target RNA.

Within the novel compositions of the invention, the siNA may be admixed or complexed with the polypeptide and one or more non-cationic lipids or a combination of a non-cationic lipid and a cationic lipid to form a composition that enhances intracellular delivery of the siNA as compared to delivery resulting from contacting the target cells with a naked siNA (i.e., siNA without a polypeptide and/or a non-cationic lipid present). The siNA may also be conjugated to the polypeptide and admixed with one or more non-cationic lipids or a combination of a non-cationic lipid and a cationic lipid to form a composition that enhances intracellular delivery of the siNA as compared to delivery resulting from contacting the target cells with a naked siNA.

Further, within the novel compositions of the invention, the siNA may be admixed or complexed with, or conjugated to, the polypeptide to form a composition that enhances intracellular delivery of the siNA as compared to delivery resulting from contacting the target cells with a naked siNA (i.e., siNA without the polypeptide present).

To produce these compositions comprised of a polypeptide, siRNA and one or more non-cationic lipids or a combination of a non-cationic lipid and a cationic lipid, the siRNA and polypeptide may be mixed together first, followed by the addition of one or more non-cationic lipids or a combination of a non-cationic and a cationic lipid added in a suitable medium such as a cell culture medium. Optionally, the polypeptide and one or more non-cationic lipids or a combination of a non-cationic lipid and a cationic lipid may be mixed first followed by the addition of the siRNA in a suitable medium such as a cell culture medium.

Within the compositions, formulations and methods of this invention, the active agent may be combined or coordinately administered with a suitable carrier or vehicle. As used herein, the term “carrier” includes a pharmaceutically acceptable solid or liquid filler, diluent or encapsulating or carrying material.

A carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients, pharmaceutical excipients and/or additives of the above categories suitable for use in the compositions and formulations of this invention can be found in the U.S. Pharmacopeia National Formulary, 1990, pp. 1857-1859, as well as in Raymond C. Rowe, et al., Handbook of Pharmaceutical Excipients, 5th ed., 2006, and “Remington: The Science and Practice of Pharmacy,” 21st ed., 2006, editor David B. Troy, and in the Physician's Desk Reference, 52nd ed., Medical Economics, Montvale, N.J., 1998.

Some examples of the materials which can serve as pharmaceutically acceptable carriers are sugars, such as monosaccharides, disaccharides and polysaccharides, including glucose, xylose, fructose, reose, ribose, pentose, arabinose, allose, tallose, altrose, mannose, galactose, lactose, sucrose, erythrose, or any combinations thereof; glyceraldehyde; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Some examples of pharmaceutically acceptable antioxidants include water soluble antioxidants such as ascorbic acid, cysteine hydrochloride, sodium bisulfite, sodium metabisulfite, and sodium sulfite; oil-soluble antioxidants such as ascorbyl palmitate, butylated hydroxyanisole (BHA), butylated hydroxytoluene (BHT), lecithin, propyl gallate, and alpha-tocopherol; and metal-chelating agents such as citric acid, ethylenediamine tetraacetic acid (EDTA), sorbitol, tartaric acid, and phosphoric acid.

### Neutral Lipids

A neutral lipid, also termed a “helper lipid,” has zero net charge at the pH where it is considered to be neutral. Most neutral lipids are zwitterionic meaning the lipid has a net zero charge as a result of the presence of both a single positive charge and single negative charge within the lipid separated by more than one atom.

Non-limiting examples of useful neutral lipids within the invention include 1,2-Dilauroyl-sn-glycerol (DLG); 1,2-Dimyristoyl-sn-glycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-sn-glycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-O-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoyl-sn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn-1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-sn-glycero-3-phospho-sn-1-glycerol (sodium salt; DSP-sn-1-G); 1,2-Dipalmitoyl-sn-glycero-3-phospho-L-serine (sodium sal; DPPS); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-sn-glycero-3-phosphoglycerol (ammonium salt; POPG); 1-Palmitoyl-2-lyso-sn-glycero-3-phosphocholine (P-lyso-PC); 1-Stearoyl-2-lyso-sn-glycero-3-phosphocholine (S-lyso-PC); N-(Carbonyl-methoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N-(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N-(Carbonyl-methoxypolyethyleneglycol 2000)-1,2-distearoyl-sn-glycero-3-phosphoethanolamine (sodium salt; DSPE-MPEG-2000); N-(Carbonyl-methoxypolyethyleneglycol 5000)-1,2-distearoyl-sn-glycero-3-phosphoethanolamine (sodium salt; DSPE-MPEG-5000); Sodium cholesteryl sulfate (SCS).

### Surfactants

Within certain embodiments of the invention, the siNA may be mixed with natural or synthetic surfactants or a combination. Natural surfactants are found in human lung (pulmonary surfactant), which is a complex mixture of phospholipids and proteins that form a monolayer at the alveolar air-liquid interface and reduces surface tension to near zero at expiration and prevents alveolar collapse. Over 90% (by weight) of pulmonary surfactant is composed of phospholipids with approximately 40-80% being DPPC and the remainder being unsaturated phosphatidylcholines POPG, POPC and phosphatidylglycerols. The remaining 10% (by weight) of surfactant is composed of plasma proteins and apoproteins, such as surface protein (SP)-A, SP-B, SP-C and SP-D. These surfactant proteins are critical to lung function and are all phospholipid membrane associated proteins. Non-limiting examples of natural surfactants that may be used in the present invention include, but are not limited to, SURVANTA™ (beractant), CUROSURF™ (poractant alfa) and INFASURF™ (calfactant). However, these surfactants do not contain the apoproteins SP-A or SP-D.

SP-A and SP-D are members of the Collectin protein family and are opsonizing proteins that bind non-host oligosaccharides. SP-B and SP-C are multimeric-amphiphilic (hydrophobic cationic) proteins and their function is critical to lung surfactant performance. By way of example, the amino acid sequence of the human SP-B is as follows:

(SEQ ID NO: 32)

NH2-FPIPLPYCWLCRALIKRIQAMIPKGALRVAVAQVCRVVPLVAGGIC
QCLAERYS
VILLDTLLGRMLPQLVCRLVLRCS

### The full-length SP-B protein has net charge +8 and 4 disulfide links.

In contrast to natural surfactants, synthetic surfactants do not contain the apoproteins (SP-A, B, C and D). However, synthetic protein surfactants may be comprised of SP analogous. Non-limiting examples of synthetic surfactants include sinapultide, which is a combination of dipalmitoylfosfatidylcholine, palmitoyloleoyl fosfatidylglycerol and palmitic acid), SURFAXIN™ (lucinactant), EXOSURF™ (colfosceril), which contains tyloxapol, DPPC and hexadecanol.

### Polypeptide

In certain embodiments of the invention, the polypeptide (peptide) consists of a hydrophobic region and a positively charged region. The hydrophobic region consists of a cluster of at least two hydrophobic residues, preferably two or more, more preferably three or more and most preferably four or more, that lie within a stretch of nine residues that excludes a positively charged residue. The positively charged region consists of a cluster of at least three positively charged residues, preferably three or more, more preferably four or more and most preferably five or more that lie within a stretch of ten residues or less.

Within additional embodiments of the invention, the polypeptide is selected or rationally designed to comprise an amphipathic amino acid sequence. For example, useful polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In other embodiments, the polypeptide may consist of a positively charged domain and have a hydrophobic region resulting from the presence of a covalently linked lipid moiety, for example a fatty acid. In other embodiments, the polypeptide has the following amino acid sequence:

| | |
|---|---|
| NH₂-KETWWETWWTEWSQPGRKKRRQRRRPPQ | (SEQ ID NO: 36) |

In yet additional detailed embodiments, the polypeptide may be pegylated to improve stability and/or efficacy, particularly in the context of in vivo administration.

In other embodiments, the polypeptide is selected to comprise a protein transduction domain or motif, and a fusogenic peptide domain or motif. A protein transduction domain is a peptide sequence that is able to insert into and preferably transit through the membrane of cells. A fusogenic peptide is a peptide that destabilizes a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF-4).

To rationally design polypeptides of the invention, a protein transduction domain is employed as a motif that will facilitate entry of the nucleic acid into a cell through the plasma membrane. In certain embodiments, the transported nucleic acid will be encapsulated in an endosome. The interior of endosomes has a low pH resulting in the fusogenic peptide motif destabilizing the membrane of the endosome. The destabilization and breakdown of the endosome membrane allows for the release of the siNA into the cytoplasm where the siNA can associate with a RISC complex and be directed to its target mRNA.

Examples of protein transduction domains for optional incorporation into polypeptides of the invention include:

| | |
|---|---|
| 1. | TAT protein transduction domain (PTD) |
| | (SEQ ID NO: 1) KRRQRRR; |
| 2. | Penetratin PTD (SEQ ID NO: 2) RQIKIWFQNRRMKWKK; |
| 3. | VP22 PTD (SEQ ID NO: 3) |
| | DAATATRGRSAASRPTERPRAPARSASRPRRPVD; |
| 4. | Kaposi FGF signal sequences (SEQ ID NO: 4) |
| | AAVALLPAVLLALLAP, and SEQ ID NO: 5) |
| | AAVLLPVLLPVLLAAP; |
| 5. | Human β3 integrin signal sequence |
| | (SEQ ID NO: 6) VTVLALGALAGVGVG; |
| 6. | gp41 fusion sequence (SEQ ID NO: 7) |
| | GALFLGWLGAAGSTMGA; |
| 7. | Caiman crocodylus Ig(v) light chain |
| | (SEQ ID NO: 8) MGLGLHLLVLAAALQGA; |
| 8. | hCT-derived peptide (SEQ ID NO: 9) |
| | LGTYTQDFNKFHTFPQTAIGVGAP; |
| 9. | Transportan (SEQ ID NO: 10) |
| | GWTLNSAGYLLKINLKALAALAKKIL; |
| 10. | Loligomer (SEQ ID NO: 11) TPPKKKRKVEDPKKKK; |
| 11. | Arginine peptide (SEQ ID NO: 12) RRRRRRR; and |
| 12. | Amphiphilic model peptide (SEQ ID NO: 13) |
| | KLALKLALKALKAALKLA. |

Examples of viral fusion peptides fusogenic domains for optional incorporation into polypeptides of the invention include:

| | |
|---|---|
| 1. | Influenza HA2 (SEQ ID NO: 14) GLFGAIAGFIENGWEG; |
| 2. | Sendai F1 (SEQ ID NO: 15) FFGAVIGTIALGVATA; |
| 3. | Respiratory Syncytial virus F1 (SEQ ID NO: 16) |
| | FLGFLLGVGSAIASGV; |
| 4. | HIV gp41 (SEQ ID NO: 17) GVFVLGFLGFLATAGS; and |
| 5. | Ebola GP2 (SEQ ID NO: 18) GAAIGLAWIPYFGPAA. |

Within yet additional embodiments of the invention, polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified in Table 1, below (see, e.g., Simpson et al., J. Biol. Chem. 278:28011-28018, 2003).

TABLE 1
Exemplary Zinc Finger Motifs of Different DNA-binding Proteins

C₂H₂ Zinc finger motif

| | . . . . \| . . . . \| | . . . . \| . . . . \| | . . . . \| . . . . \| | . . . . \| . . . . \| | . . . . \| . . . . \| | . . . . \| . . . . \| |
|---|---|---|---|---|---|---|
| | 665 | 675 | 685 | 695 | 705 | 715 |
| Sp1 | ACTCPYCKDS | EGRGSG---- | DPGKKKQHIC | HIQGCGKVYG | KTSHLRAHLR | WHTGERPFMC |
| Sp2 | ACTCPNCKDG | EKRS------ | GEQGKKKHVC | HIPDCGKTFR | KTSLLRAHVR | LHTGERPFVC |
| Sp3 | ACTCPNCKEG | GGRGTN---- | -LGKKKQHIC | HIPGCGKVYG | KTSHLRAHLR | WHSGERPFVC |
| Sp4 | ACSCPNCREG | EGRGSN---- | EPGKKKQHIC | HIEGCGKVYG | KTSHLRAHLR | WHTGERPFIC |
| DrosBtd | RCTCPNCTNE | MSGLPPIVGP | DERGRKQHIC | HIPGCERLYG | KASHLKTHLR | WHTGERPFLC |
| DrosSp | TCDCPNCQEA | ERLGPAGV-- | HLRKKNIHSC | HIPGCGKVYG | KTSHLKAHLR | WHTGERPFVC |
| CeT22C8.5 | RCTCPNCKAI | KHG------- | DRGSQHTHLC | SVPGCGKTYK | KTSHLRAHLR | KHTGDRPFVC |
| Y40B1A.4 | PQISLKKKIF | FFIFSNFR-- | GDGKSRIHIC | HL--CNKTYG | KTSHLRAHLR | GHAGNKPFAC |

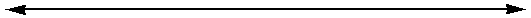

Prosite pattern
C-x(2,4)-C-x(12)-H-x(3)-H (SEQ ID NO: 37)
*Table 1 demonstrates a conservative zinc fingerer motif for double strand DNA binding which is characterized by the C-x(2,4)-C-x(12)-H-x(3)-H (SEQ ID NO: 37) motif pattern, which itself can be used to select and design additional polypeptides according to the invention.
**The sequences shown in Table 1, for Sp1, Sp2, Sp3, Sp4, DrosBtd, DrosSp, CeT22C8.5, and Y4pB1A.4, are herein assigned SEQ ID NOs: 19, 20, 21, 22, 23, 24, 25, and 26, respectively.

Alternative DNA binding domains useful for constructing polypeptides of the invention include, for example, portions of the HIV Tat protein sequence.

Within exemplary embodiments of the invention described herein below, polypeptides may be rationally designed and constructed by combining any of the foregoing structural elements, domains or motifs into a single polypeptide effective to mediate enhanced delivery of siNAs into target cells. For example, a protein transduction domain of the TAT polypeptide was fused to the N-terminal 20 amino acids of the influenza virus hemagglutinin protein, termed HA2, to yield one exemplary polypeptide herein. Various other polypeptide constructs are provided in the instant disclosure, evincing that the concepts of the invention are broadly applicable to create and use a diverse assemblage of effective polypeptides for enhancing siNA delivery.

Yet additional exemplary polypeptides within the invention may be selected from the following peptides: WWETWKPFQCRICMRNFSTRQARRNHRRRHR (SEQ ID NO: 27); GKINLKALAALAKKIL (SEQ ID NO: 28), RVIRVWFQNKRCKDKK (SEQ ID NO: 29), GRKKRRQRRRPPQGRKKRRQRRRPPQGRKKRRQRRRPPQ (SEQ ID NO: 30), GEQIAQLIAGYIDIILKKKKSK (SEQ ID NO: 31), Poly Lys-Trp, 4:1, MW 20,000-50,000; and Poly Orn-Trp, 4:1, MW 20,000-50,000. Additional polypeptides that are useful within the compositions and methods herein comprise all or part of the mellitin protein sequence.

Still other exemplary polypeptides are identified in the examples below. Any one or combination of these peptides may be selected or combined to yield effective polypeptide reagents to induce or facilitate intracellular delivery of siNAs within the methods and compositions of the invention.

In other detailed embodiments, the siNA that is admixed, complexed, or conjugated with the polypeptide will comprise a double-stranded RNA, for example a double-stranded RNA that has 30 or fewer nucleotides, and is a short interfering RNA (siRNA).

### Cationic Lipids:

A cationic lipid is generally composed of a cationic head, a linker region and a hydrophobic moiety. The hydrophobic moiety provides self-association properties of the cationic lipid allowing it to form micelles or liposomes.

In more detailed aspects of the invention, the mixture, complex or conjugate comprising a siRNA and a polypeptide can be optionally combined with (e.g., admixed or complexed with) a cationic lipid, such as LIPOFECTIN®. To produce these compositions comprised of a polypeptide, siRNA and a cationic lipid, the siRNA and peptide may be mixed together first in a suitable medium such as a cell culture medium, after which the cationic lipid is added to the mixture to form a siRNA/delivery peptide/cationic lipid composition. Optionally, the peptide and cationic lipid can be mixed together first in a suitable medium such as a cell culture medium, whereafter the siRNA can be added to form the siRNA/delivery peptide/cationic lipid composition.

Non-limiting examples of useful cationic lipids within the invention include N-[1-(2,3-dioleoyloxy)propyl]-N,N,N-trimethylammonium chloride, 1,2-bis(oleoyloxy)-3-3-(trimethylammonium)propane, 1,2-dimyristyloxypropyl-3-dimethylhydroxyethylammonium bromide, and dimethyldioctadecylammonium bromide, 2,3-dioleyloxy-N-[2(sperminecarboxamido)ethyl]-N,N-dimethyl-1-propanaminium trifluoracetate, 1,3-dioleoyloxy-2-(6-carboxyspermyl)-propylamid, 5-carboxyspermylglycine dioctadecylamide, tetramethyltetrapalmitoyl spermine, tetramethyltetraoleyl spermine, tetramethyltetralauryl spermine, tetramethyltetramyristyl spermine and tetramethyldioleyl spermine. DOTMA (N-[1-(2,3-dioleoyloxy)propyl]-N,N,N-trimethyl ammonium chloride), DOTAP (1,2-bis(oleoyloxy)-3,3-(trimethylammonium)propane), DMRIE (1,2-dimyristyloxypropyl-3-dimethyl-hydroxy ethyl ammonium bromide) or DDAB (dimethyl dioctadecyl ammonium bromide). Polyvalent cationic lipids include lipospermines, specifically DOSPA (2,3-dioleyloxy-N-[2(sperminecarboxamido)ethyl]-N,N-dimethyl-1-propanaminium trifluoro-acetate) and DOSPER (1,3-dioleoyloxy-2-(6carboxy spermyl)-propyl-amid, and the di- and tetra-alkyl-tetra-methyl spermines, including but not limited to TMTPS (tetramethyltetrapalmitoyl spermine), TMTOS (tetramethyltetraoleyl spermine), TMTLS (tetramethlytetralauryl spermine), TMTMS (tetramethyltetramyristyl spermine) and TMDOS (tetramethyldioleyl spermine) DOGS (dioctadecyl-amidoglycylspermine (TRANSFECTAM®). Other useful cationic lipids are described, for example, in U.S. Pat. No. 6,733,777; U.S. Pat. No. 6,376,248; U.S. Pat. No. 5,736,392; U.S. Pat. No. 5,686,958; U.S. Pat. No. 5,334,761 and U.S. Pat. No. 5,459,127.

Cationic lipids are combined with non-cationic lipids, particularly neutral lipids, for example lipids such as DOPE (dioleoylphosphatidylethanolamine), DPhPE (diphytanoylphosphatidylethanolamine) or cholesterol. A cationic lipid composition composed of a ratio of non-cationic lipid to cationic lipid including 1:1; 1:2; 1:3; 2:1 and 3:1.

Preferred transfection compositions are those which induce substantial transfection of a higher eukaryotic cell line.

In exemplary embodiments, the instant invention features compositions comprising a small nucleic acid molecule, such as short interfering nucleic acid (siNA), a short interfering RNA (siRNA), a double-stranded RNA (dsRNA), micro-RNA (mRNA), or a short hairpin RNA (shRNA), admixed or complexed with the polypeptide and one or more non-cationic lipids or a combination of a non-cationic lipid and a cationic lipid to form a composition that enhances intracellular delivery of the siNA as compared to delivery resulting from contacting the target cells with a naked siNA (i.e., siNA without a polypeptide and/or a non-cationic lipid present). The siNA may also be conjugated to the polypeptide and admixed with one or more non-cationic lipids or a combination of a non-cationic lipid and a cationic lipid to form a composition that enhances intracellular delivery of the siNA as compared to delivery resulting from contacting the target cells with a naked siNA.

Further, within the novel compositions of the invention, the siNA may be admixed or complexed with, or conjugated to, the polypeptide to form a composition that enhances intracellular delivery of the siNA as compared to delivery resulting from contacting the target cells with a naked siNA (i.e., siNA without the polypeptide present).

### Definitions

The term “phospholipids” means a lipid that in its simplest form is composed of glycerol bonded to two fatty acids and a phosphate group. The resulting compound called phosphatidic acid contains a region (the fatty acid component) that is fat-soluble along with a region (the charged phosphate group) that is water-soluble. Most phospholipids also have an additional chemical group bound to the phosphate. For example, it may be connected with choline; the resulting phospholipid is called phosphatidylcholine, or lecithin. Other phospholipids include phosphatidylglycerol, phosphatidylinositol, phosphatidylserine, and phosphatidylethanolamine. The bipolar character of phospholipids is essential to their biological function in cell membranes. The fat-soluble portions associate with the fat-soluble portions of other phospholipids while the water-soluble regions remain exposed to the surrounding solvent. The phospholipids of the cell membrane form into a sheet two molecules thick with the fat-soluble portions inside shielded on both sides by the water-soluble portions. This stable structure provides the cell membrane with its integrity.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. (C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkylene” by itself or as part of another substituent means a divalent radical derived from an alkane, as exemplified by —CH₂CH₂CH₂CH₂—. Typically, an alkyl or alkylene group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “sugar unit” as used herein relates to a monosaccharide or it can relate to a polysaccharide. Examples of monosaccharides for use within the invention include, but are not limited to the D- and L-chiral forms of: arabinose, allose, altrose, erythrose, threose, galactose, glucose, gulose, fructose, idose, lyxose, mannose, ribose, threose, ribulose, tagatose, talose, 2-deoxyribose, and xylose. Examples of polysaccharides for use within the invention include, but are not limited to any combination of two or more monosaccharides.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, for example a siRNA, that is a short-length double-stranded nucleic acid (or optionally a longer precursor thereof), and which is not unacceptably toxic in target cells. The length of useful siNAs within the invention will in certain embodiments be optimized at a length of approximately 21 to 23 base pairs (bp) long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within the invention will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

In certain embodiments of the invention, as noted above, polypeptides and one or more non-cationic lipids or a combination of a non-cationic lipid and a cationic lipid are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell. For example, a siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

### Small-Interfering Nucleic Acids and the RISC Complex

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present invention avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing two nucleotide 3′-overhangs. Furthermore, complete substitution of one or both siRNA strands with 2′-deoxy(2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other cause are also contemplated. Furthermore, in the present invention the double-stranded region of siNAs in which two strands pair up may contain both bulge and mismatched portions in the approximate numerical ranges specified.

The terminal structure of siNAs of the invention may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example, Martinez et al., Cell. 110:563-574, 2002, and Schwarz et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of the invention lack 2′-hydroxy(2′-OH) containing nucleotides. In certain embodiments short interfering nucleic acids do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within the invention may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic interactions, and/or stacking interactions.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. Optionally, the siRNA include single strands or double strands of siRNA.

An siHybrid molecule is a double-stranded nucleic acid that has a similar function to siRNA. Instead of a double-stranded RNA molecule, an siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′overhanging end.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of the invention can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of a siNA molecule of the invention can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of the invention can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′- and 5′-ends of the sense strand, the antisense strand, or both strands. For example, an exemplary siNA molecule of the invention can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, the invention features siNA molecules including modified nucleotides having a Northern conformation (e.g., Northern pseudorotation cycle, see for example, Saenger, “Principles of Nucleic Acid Structure,” Springer-Verlag ed., 1984). As such, chemically modified nucleotides present in the siNA molecules of the invention, preferably in the antisense strand of the siNA molecules of the invention, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, ribothymidine nucleotides and 2′-O-methyl nucleotides.

The sense strand of a double stranded siNA molecule may have a terminal cap moiety such as an inverted deoxybasic moiety, at the 3′-end, 5′-end, or both 3′ and 5′-ends of the sense strand.

Non-limiting examples of conjugates include conjugates and ligands described in Vargeese et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof. In one embodiment, a conjugate molecule of the invention comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of the invention can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

A siNA further may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. [See, for example, Gold et al, Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jschke et al., Tetrahedron Lett. 34:301, 1993; Ono et al., Biochemistry 30:9914, 1991; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 113:4000, 1991. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymidine, for example at the C1 position of the sugar.

The synthesis of a siNA molecule of the invention, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers et al., Methods in Enzymology 211:3-19, 1992; Thompson et al., International PCT Publication No. WO 99/54459; Wincott et al., Nucleic Acids Res. 23:2677-2684, 1995; Wincott et al., Methods Mol. Bio. 74:59, 1997; Brennan et al., Biotechnol Bioeng. 61:33-45, 1998; and Brennan, U.S. Pat. No. 6,001,311. Synthesis of RNA, including certain siNA molecules of the invention, follows general procedures as described, for example, in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995; Wincott et al., Methods Mol. Bio. 74:59, 1997.

Supplemental or complementary methods for delivery of nucleic acid molecules for use within then invention are described, for example, in Akhtar et al., Trends Cell Bio. 2:139, 1992; Delivery Strategies for Antisense Oligonucleotide Therapeutics, ed. Akhtar, 1995; Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Sullivan et al., International PCT Publication No. WO 94/02595, further describes general methods for delivery of enzymatic nucleic acid molecules. These protocols can be utilized to supplement or complement delivery of virtually any nucleic acid molecule contemplated within the invention.

Nucleic acid molecules and polypeptides can be administered to cells by a variety of methods known to those of skill in the art, including, but not restricted to, administration within formulations that comprise the siNA and polypeptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262.

The compositions of the instant invention can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments the invention provides pharmaceutical compositions and methods featuring the presence or administration of one or more polynucleic acid(s), typically one or more siNAs, combined, complexed, or conjugated with a polypeptide, optionally formulated with a pharmaceutically-acceptable carrier, such as a diluent, stabilizer, buffer, and the like.

The present invention satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of the invention may be targeted to lower expression of one gene, which can result in upregulation of a “downstream” gene whose expression is negatively regulated by a product or activity of the target gene.

Within exemplary embodiments, the compositions and methods of the invention are useful as therapeutic tools to regulate expression of tumor necrosis factor-α (TNF-α) to treat or prevent symptoms of rheumatoid arthritis (RA). In this context the invention further provides compounds, compositions, and methods useful for modulating expression and activity of TNF-α by RNA interference (RNAi) using small nucleic acid molecules. In more detailed embodiments, the invention provides small nucleic acid molecules, such as short interfering nucleic acid (siNA), short interfering RNA (siRNA), double-stranded RNA (dsRNA), micro-RNA (mRNA), and short hairpin RNA (shRNA) molecules, and related methods, that are effective for modulating expression of TNF-α and/or TNF-α genes to prevent or alleviate symptoms of RA in mammalian subjects. Within these and related therapeutic compositions and methods, the use of chemically-modified siNAs will often improve properties of the modified siNAs in comparison to properties of native siNA molecules, for example by providing increased resistance to nuclease degradation in vivo, and/or through improved cellular uptake. As can be readily determined according to the disclosure herein, useful siNAs having multiple chemical modifications will retain their RNAi activity. The siNA molecules of the instant invention thus provide useful reagents and methods for a variety of therapeutic, diagnostic, target validation, genomic discovery, genetic engineering, and pharmacogenomic applications.

This siNAs of the present invention may be administered in any form, for example transdermally or by local injection (e.g., local injection at sites of psoriatic plaques to treat psoriasis, or into the joints of patients afflicted with psoriatic arthritis or RA). In more detailed embodiments, the invention provides formulations and methods to administer therapeutically effective amounts of siNAs directed against of a mRNA of TNF-α, which effectively down-regulate the TNF-α RNA and thereby reduce or prevent one or more TNF-α-associated inflammatory condition(s). Comparable methods and compositions are provided that target expression of one or more different genes associated with a selected disease condition in animal subjects, including any of a large number of genes whose expression is known to be aberrantly increased as a causal or contributing factor associated with the selected disease condition.

The siNA/polypeptide one or more non-cationic lipids or a combination of a non-cationic lipid and a cationic lipid mixtures of the invention can be administered in conjunction with other standard treatments for a targeted disease condition, for example in conjunction with therapeutic agents effective against inflammatory diseases, such as RA or psoriasis. Examples of combinatorially useful and effective agents in this context include non-steroidal antiinflammatory drugs (NSAIDs), methotrexate, gold compounds, D-penicillamine, the antimalarials, sulfasalazine, glucocorticoids, and other TNF-α neutralizing agents such as infliximab and entracept.

Negatively charged polynucleotides of the invention (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS [Jolliet-Riant and Tillement, Fundam. Clin. Pharmacol. 13:16-26, 1999]; biodegradable polymers, such as poly(DL-lactide-coglycolide) microspheres for sustained release delivery after intracerebral implantation (Emerich, D. F. et al., Cell Transplant 8:47-58, 1999, Alkermes, Inc., Cambridge, Mass.; and loaded nanoparticles, such as those made of polybutylcyanoacrylate, which can deliver drugs across the blood brain barrier and can alter neuronal uptake mechanisms (Prog. Neuropsychopharmacol Biol Psychiatry 23:941-949, 1999). Other non-limiting examples of delivery strategies for the nucleic acid molecules of the instant invention include material described in Boado et al., J. Pharm. Sci. 87:1308-1315, 1998; Tyler et al., FEBS Lett. 421:280-284, 1999; Pardridge et al., PNAS USA. 92:5592-5596, 1995; Boado, Adv. Drug Delivery Rev. 15:73-107, 1995; Aldrian-Herrada et al., Nucleic Acids Res. 26: 4910-4916, 1998; and Tyler et al., PNAS USA. 96:7053-7058, 1999.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co., A. R. Gennaro ed. 1985. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The siNAs can be modified extensively to enhance stability by modification with nuclease resistant groups, for example, 2′-amino, 2′-C-allyl, 2′-fluoro, 2′-O-methyl, 2′-H. [For a review see Usman and Cedergren, TIBS 17:34, 1992; Usman et al., Nucleic Acids Symp. Ser. 31:163, 1994. SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., Eckstein et al., International Publication No. WO 92/07065; Perrault et al., Nature 344:565, 1990; Pieken et al., Science 253:314, 1991; Usman and Cedergren, Trends in Biochem. Sci. 17:334, 1992; Usman et al., International Publication No. WO 93/15187; and Rossi et al., International Publication No. WO 91/03162; Sproat, U.S. Pat. No. 5,334,711; Gold et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman et al., Nucleic Acids Symp. Ser. 31:163, 1994; Burgin et al., Biochemistry 35:14090, 1996. Sugar modification of nucleic acid molecules have been extensively described in the art. See Eckstein et al., International Publication PCT No. WO 92/07065; Perrault et al., Nature 344:565-568, 1990; Pieken et al. Science 253:314-317, 1991; Usman and Cedergren, Trends in Biochem. Sci. 17:334-339, 1992; Usman et al. International Publication PCT No. WO 93/15187; Sproat, U.S. Pat. No. 5,334,711, and Beigelman et al., J. Biol. Chem. 270:25702, 1995; Beigelman et al., International PCT Publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., Karpeisky et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina et al., Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant invention so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties, in Modern Synthetic Methods,” VCH, 1995, 331-417, and Mesmaeker et al., “Novel Backbone Replacements for Oligonucleotides, in Carbohydrate Modifications in Antisense Research,” ACS, 1994, 24-39.

Methods for the delivery of nucleic acid molecules are described in Akhtar et al., Trends Cell Bio. 2:139, 1992; Delivery Strategies for Antisense Oligonucleotide Therapeutics, ed. Akhtar, 1995; Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez et al., Bioconjugate Chem. 10:1068-1074, 1999; Wang et al., International PCT Publication Nos. WO 03/47518 and WO 03/46185), poly(lactic-co-glycolic)ac-id (PLGA) and PLCA microspheres (see for example U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

The term “ligand” refers to any compound or molecule, such as a drug, peptide, hormone r neurotransmitter that is capable of interacting with another compound, such as a receptor, either directly or indirectly. The receptor that interacts with a ligand can be present on the surface of a cell or can alternately be an intracellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of the invention can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a T-cell (e.g. about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition.

By “inhibit”, “down-regulate”, “knockdown” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant invention is greater in the presence of the nucleic acid molecule than in its absence.

Gene “silencing” refers to partial or complete loss-of-function through targeted inhibition of gene expression in a cell and may also be referred to as “knockdown.” Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of the invention, including prophylactic and therapeutic methods, which will be capable of knocking down target gene expression, in terms of mRNA levels or protein levels or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “subject” is meant an organism, tissue, or cell, which may include an organism as the subject or as a donor or recipient of explanted cells or the cells that are themselves subjects for siNA delivery. “Subject” therefore may refers to an organism, organ, tissue, or cell, including in vitro or ex vivo organ, tissue or cellular subjects, to which the nucleic acid molecules of the invention can be administered and enhanced by polypeptides described herein. Exemplary subjects include mammalian individuals or cells, for example human patients or cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

By “vectors” is meant any nucleic acid- and/or viral-based technique used to deliver a desired nucleic acid.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “RNA” is meant a molecule comprising at least one ribonucleotide residue. By “ribonucleotide” is meant a nucleotide with a hydroxyl group at the 2′ position of a beta.-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “target nucleic acid” or “nucleic acid target” or “target RNA” or “RNA target” or “target DNA” or “DNA target” is meant any nucleic acid sequence whose expression or activity is to be modulated. The target nucleic acid can be DNA or RNA and is not limited single strand forms.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII:123-133, 1987; Frier et al., Proc. Nat. Acad. Sci. USA 83:9373-9377, 1986; Turner et al., J. Am. Chem. Soc. 109:3783-3785, 1987. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, Nucleic Acids Research 29:2437-2447, 2001).

The term “acyclic nucleotide” as used herein refers to any nucleotide having an acyclic ribose sugar, for example where any of the ribose carbons (C₁, C2, C3, C4, or C5), are independently or in combination absent from the nucleotide.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, Tetrahedron 49:1925, 1993; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., Biochemistry 35:14090, 1996; Uhlman & Peyman, supra). By “modified bases” in this aspect is meant nucleotide bases other than adenine, guanine, cytosine and uracil at 1′ position or their equivalents.

By “target site” or “target sequence” or “targeted sequence” is meant a sequence within a target nucleic acid (e.g., RNA) that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi activity. The term “biological system” includes, for example, a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, for example, a biologically active molecule to a siNA molecule of the invention or the sense and antisense strands of a siNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of beta.-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., U.S. Pat. No. 6,248,878.

The siNA molecules can be complexed with cationic lipids, packaged within liposomes, or otherwise delivered to target cells or tissues. The nucleic acid or nucleic acid complexes can be locally administered to through injection, infusion pump or stent, with or without their incorporation in biopolymers. In another embodiment, polyethylene glycol (PEG) can be covalently attached to siNA compounds of the present invention, to the polypeptide, or both. The attached PEG can be any molecular weight, preferably from about 2,000 to about 50,000 daltons (Da).

The sense region can be connected to the antisense region via a linker molecule, such as a polynucleotide linker or a non-nucleotide linker.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Large double-stranded RNA” refers to any double-stranded RNA having a size greater than about 40 bp for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of sequence.

“Target gene or mRNA” refers to any gene or mRNA of interest. Indeed any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. Such cells include any cell in the body of an adult or embryonic animal or plant including gamete or any isolated cell such as occurs in an immortal cell line or primary cell culture.

In this specification and the appended claims, the singular forms of “a”, “an” and “the” include plural reference unless the context clearly dictates otherwise.

### EXAMPLES

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Materials and Methods Used

The present example illustrates the materials and methods used to assess the efficacy of the one or more non-cationic lipids or a combination of a non-cationic lipid and a cationic lipid and polypeptide mixture to enhance siRNA cell-uptake and permit siRNA mediated target gene knockdown activities in vitro. Cell viability was also assessed. The cell culture conditions and protocols for each assay are explained below in detail.

### Materials

Table 2 below shows the materials and their source used in the instant application. Both the LacZ siRNA (gactacacaaatcagcgattt (SEQ ID NO: 33)) and Qneg siRNA (uucuccgaacgugucacgu (SEQ ID NO: 34)) were from QIAGEN™. The Qneg siRNA served as a negative control and was labeled with Alexa 546 at the 3′-end of sense strand. The 9L/LacZ cell line was obtained from the American Type Culture Collection (ATCC™). The amino acid sequence of the polynucleotide-delivery enhancing peptide PN73 is as follows:

(SEQ ID NO: 35)

NH₂-KGSKKAVTKAQKKDGKKRKRSRKESYSVYVYKVLKQ

and the amino acid sequence of the polynucleotide-delivery enhancing peptide PN183 is as follows:

(SEQ ID NO: 36)

NH₂- KETWWETWWT EWSQPGRKKR RQRRRPPQ

TABLE 2
Materials and Source

| Reagent | Source |
|---|---|
| LacZ siRNA | QIAGEN ™ |
| Qneg Alexa546 siRNA | QIAGEN ™ |
| (negative control) | |
| 9L/LacZ Cell Line | ATCC ™ |
| PN73 Peptide | NASTECH PHARMACEUTICAL INC. ™ |
| PN183 Peptide | NASTECH PHARMACEUTICAL INC. ™ |
| DMEM Cell Media | MEDIATECH, INC ™ |
| (CELLGRO ™) | |
| Fetal Bovine Serum | HYCLONE ™ |
| HiPerFect | QIAGEN ™ |
| DOPE | AVENTI POLOR LIPIDS ™ |
| CHEMS | SIGMA-ALDRICH ™ |
| Cholesterol | SIGMA-ALDRICH ™ |

### Cell Cultures

The 9L/LacZ cell line constitutively expresses LacZ. 9L/LacZ cells were propagated in Dulbecco's modified Eagle's medium (DMEM) with 4.5 g/L glucose, 1 mM sodium pyruvate 90% and 10% fetal bovine serum at 37° C., 5% CO₂.

### Lipid Stocks

The lipids were transferred to glass vials with TEFLON™ caps and the chloroform or methanol was removed by evaporation with a stream of argon gas. The resulting thin lipid film on the wall of the glass vial was vacuum dried overnight. The following day, the lipids were resuspended in 10 mM HEPES buffer (pH 7.4) by vortexing and subject to three freeze-thaw cycles in a dry ice ethanol bath. The resuspended lipids were stored overnight at 4° C. Prior to use, the lipid suspension was sonicated for five minutes in a 65° C. water bath. The working stock concentration of each lipid was 1 mg/ml.

### LacZ Assay

The LacZ assay was performed three days post-transfection. Cells were rinsed with phosphate buffers saline (PBS) after removal of cell media. The PBS was aspirated and the cells were lysed by the addition of 50 μl M-PER™ to each well and incubating the cells for 15 minutes while gently shaking. Following cell lysis, 10 μl of cell lysate was removed from each well to perform a total protein assay (micro BCA kit, Pierce). In order to measure LacZ activity, 30 μl of cell lysate was transferred to a new plate and 30 μl of ALL-IN-ONE™ beta-galactosidase (β-gal) was added to each well of the new plate. The new plate was covered to avoid light exposure and incubated for 30 minutes at 37° C. Following the 30 minute incubation, the absorbance was read at 405 nM with a μQuant plate reader (Bio-Trek Instrument) to measure LacZ activity.

### Sephadex CL-4B Size Exclusion Separation

Peptide Synthesis

Peptides were synthesized by solid-phase Fmoc chemistry on CLEAR-amide resin using a Rainin Symphony synthesizer. Coupling steps were performed using five equivalents of HCTU and Fmoc amino acid with an excess of N-methylmorpholine for 40 minutes. Fmoc removal was accomplished by treating the peptide resin with 20% piperidine in DMF for two 10 minutes cycles. Upon completion of the entire peptide, the Fmoc group was removed with piperidine and washed extensively with DMF. Maleimido modified peptides were prepared by coupling 3.0 equivalents of 3-maleimidopropionic acid and HCTU in the presence of six equivalents of N-methylmorpholine to the N-terminus of the peptide resin. The extent of coupling was monitored by the Kaiser test. The peptides were cleaved from the resin by the addition of 10 mL of TFA containing 2.5% water and 2.5 triisopropyl silane followed by gentle agitation at room temperature for two hours. The resulting crude peptide was collected by trituration with ether followed by filtration. The crude product was dissolved in Millipore water and lyophilized to dryness. The crude peptide was taken up in 15 mL of water containing 0.05% TFA and 3 mL acetic acid and loaded onto a Zorbax RX-C8 reversed-phase (22 mm ID×250 mm, 5 μm particle size) through a 5 mL injection loop at a flow rate of 5 mL/min. The purification was accomplished by running a linear AB gradient of 0.1% B/min where solvent A is 0.05% TFA in water and solvent B is 0.05% TFA in acetonitrile. The purified peptides were analyzed by HPLC and ESMS.

### Example 2

### Efficient Cellular Uptake of siRNA in the Presence of a Non-Cationic Lipid and Polypeptide

The present example demonstrates that efficient siRNA cellular uptake is achieved when the siRNA is combined with a non-cationic lipid and an exemplary polypeptide. The transfection efficiency of siRNA achieved with anionic lipids, neutral lipids and a combination of an anionic and/or neutral lipid with the exemplary polypeptide was compared. Further, the effect of each transfection condition on cell growth was also analyzed.

For the instant example, the Qneg siRNA was conjugated with the fluorescent tag Alexa 546 at the 3′-end of the sense strand in order to visualize its cellular location by microscopy. Table 3 below illustrates the different lipids used alone or in combination to transfect siRNA. If different lipids were used in combination, their respective ratio is also shown. In the instant example, DOPE (1,2-Dioleoyl-sn-Glycero-3-Phosphoethanolamine) and Cholesterol were used as neutral lipids. CHEMS (Cholesterol hemisuccinate salt) was used as an anionic lipid.

TABLE 3
Non-Cationic Lipids Used to Transfect siRNA

| Group | Lipids | Lipid Charge | Ratio |
|---|---|---|---|
| L1 | DOPE | Neutral | |
| L2 | DOPE:CHEMS | Neutral:Charged | 3:1 |
| L3 | CHEMS | Charged | |
| L4 | Cholesterol | Neutral | |
| L5 | DOPE:Cholesterol | Neutral | 3:1 |
| L6 | DOPE:CHEMS | Neutral:Charged | 1:1 |
| L7 | DOPE:Cholesterol | Neutral | 1:1 |

The fluorescent conjugated Qneg siRNA was transfected with one of the above lipid groups (L1 through L7) or one of the polypeptides PN73 or PN183 in combination with one of the lipid groups (L1 through L7). Transfections of siRNA were also performed with the polypeptide PN73 or PN183 alone. For each transfection mixture, 9L/LacZ cells (passage 8) were plated at density of 6.0×10³ cells/well on a 96-well plate. For the instant example, the Qneg siRNA was transfected at 100 nM with 20 nM or 60 nM of one of the lipid groups from Table 3 alone or in combination with 2 μM of the polypeptide PN73 or 7 μM of the polypeptide PN183. The cationic lipid HIPERFECT™ served as a positive transfection control. Non-transfected cells served as a negative transfection control and a positive control for cell growth.

The Qneg siRNA transfection with polypeptide alone was performed by adding 0.5 μl of Qneg siRNA stock solution (20 μM) to 12.5 μl Opti-MEM and vortexing. In a separate microcentrifuge tube, 0.2 μl of either PN73 or PN183 was mixed with 12.5 μl Opti-MEM. The contents of each tube were combined and vortexed for approximately 10 seconds and then incubated for 5-10 minutes at room temperature. The cells on the 96-well plate were rinsed once with Opti-MEM and then fed 75 μl Opti-MEM. The 25 μl transfection mixture was then added to each well and the cells were allowed to incubate with the transfection mixture for approximately three hours at 37° C., 5% CO. Fresh media was then added to each well and the cells were allowed to recover for 72 hours. At 24 hours post-transfection, the cell media was replaced with fresh cell media. Cells were visualized by microscopy with a TRIC filter and transmission light.

The Qneg siRNA transfection of 9L/LacZ cells with one of the lipid groups shown in Table 3 was performed in a similar manner to the polypeptide alone transfection protocol described above. The transfected cells were also visualized by microscopy with a TRIC filter and transmission light.

The Qneg siRNA transfection of 9L/LacZ cells with the polypeptide and lipid combination was performed in a similar manner to the transfection protocol described above, but with slight modification. Briefly, the polypeptide PN73 (2 μM) or PN183 (7 μM) was combined with one of the lipid groups from Table 3 at 20 nM or 60 nM and incubated for five minutes at room temperature. The Qneg siRNA (100 nM) was then added to the polypeptide/lipid combination. The cells were incubated with the transfection mixture for three hours at 37° C., 5% CO, and then 100 μl complete media was added. The cells were allowed to recover for 72 hours with a change of fresh complete media 24 hours post-transfection. Cells were visualized by microscopy with a TRIC filter and transmission light.

The transfection efficiency and cell growth results for the instant example are shown in Table 4. Transfection efficiency was calculated by dividing the number of fluorescently labeled cells by the total number of cells and multiplying by 100. Cell confluency was used to measure the effect of each transfection condition on cell growth. The results show that the lipids in groups L1 through L7 at 20 nM and 60 nM fail to transfect 9L/LacZ cells with the Qneg siRNA. In contrast, a high transfection efficiency of Qneg siRNA is achieved when the lipids in groups L1 through L7 at 20 nM and 60 nM were combined with the polypeptide PN183. A similar result was observed when the lipids in groups L1 through L7 were combined with the polypeptide PN73. Additionally, both polypeptides, in the absence of lipids, transfected 9L/LacZ cells with Qneg siRNA with high efficiency. In general, the lipids in groups L1, L2 and L4 through L7 alone or in combination with a polypeptide had little effect on cell growth compared to the non-treated control cells. Both the L3 lipid group and HYPERFECT™ prevented cell growth.

TABLE 4
Transfection Efficiency and Cell Growth

| | | |
|---|---|---|
| | % Fluorescent | % Cell |
| Transfection | Cells | Confluency |

| Mixture | 60 nM | 20 nM | 60 nM | 20 nM |
|---|---|---|---|---|
| Nontransfected | N/A | N/A | 70% | |
| L1 | 0% | 0% | 70% | 70% |
| L2 | 0% | 0% | 70% | 70% |
| L3 | 0% | 0% | 0% | 40% |
| L4 | 0% | 0% | 70% | 70% |
| L5 | 0% | 0% | 70% | 70% |
| L6 | 0% | 0% | 60% | 70% |
| L7 | 0% | 0% | 70% | 70% |
| HIPERFECT ™ | 0% | 0% | 0% | 0% |
| L1 + PN183 | 100% | 100% | 70% | 70% |
| L2 + PN183 | 100% | 100% | 70% | 30% |
| L3 + PN183 | 100% | 100% | 50% | 32% |
| L4 + PN183 | 100% | 100% | 70% | 60% |
| L5 + PN183 | 100% | 100% | 70% | 70% |
| L6 + PN183 | 100% | 100% | 60% | 60% |
| L7 + PN183 | 100% | 100% | 60% | 60% |
| HIPERFECT ™ | 100% | 100% | 0% | 0% |
| PN183 | 100% | 100% | 70% | 70% |
| L1 + PN73 | 100% | 100% | 70% | 70% |
| L2 + PN73 | 100% | 100% | 70% | 70% |
| L3 + PN73 | 100% | 100% | 0% | 0% |
| L4 + PN73 | 100% | 100% | 70% | 70% |
| L5 + PN73 | 100% | 100% | 70% | 70% |
| L6 + PN73 | 100% | 100% | 40% | 70% |
| L7 + PN73 | 100% | 100% | 60% | 70% |
| HIPERFECT ™ | 0% | 100% | 0% | 0% |
| PN73 | 10% | 100% | 70% | 70% |

These data in Table 4 show that highly efficient siRNA cellular uptake can be achieved when siRNA is combined with a non-cationic lipid and polypeptide.

### Example 3

### Effective In Vitro Knockdown of β-Galactosidase Activity by a LacZ siRNA Transfected with an Exemplary Polypeptide and Non-Cationic Lipid Delivery Vehicle

The present example demonstrates that siRNA transfected with one or more non-cationic lipids and exemplary polypeptide combination effectively reduces the activity of the gene product of the transcript targeted for degradation by the siRNA in vitro. This result is in contrast to the failure of the siRNA to knockdown the activity of the gene product of the transcript targeted for degradation by the siRNA when the siRNA is transfected with one or more non-cationic lipids without the exemplary polypeptide.

The present example is distinguished from prior Example 2 in that it describes transfection conditions that permit efficient functionality of siRNA (i.e., mediated degradation of a target RNA) within the cell after intracellular delivery (transfection). The prior Example 2 measured the ability of the siRNA to enter the cell (transfection efficiency or uptake). Thus, high transfection efficiency without functionality renders the delivery of a siRNA for a desired effect mute. The present example demonstrates that the combination of one or more non-cationic lipids and an exemplary polypeptide delivers siRNA within the cell and permits significant reduction of siRNA targeted transcripts as measured by a significant reduction in the gene product of the targeted transcript.

For the purpose of the instant example, the level of measured LacZ activity was correlated with the quantity of LacZ transcript within 9L/LacZ cells. Thus, a reduction in LacZ activity after siRNA transfection, without having a negative impact on cell viability, was attributed to a reduction in the quantity of LacZ transcripts via targeted degradation mediated by the LacZ siRNA.

The lipid groups shown in Table 3 of Example 2 were used at a concentration of 7 nM, 20 nM and 60 nM alone or in combination with the polypeptide PN73 at 2 μM or the polypeptide PN183 at 7 μM to transfect 9L/LacZ cells with siRNA in the instant example. The same general transfection protocol described in Example 2 was used in the instant example. The Qneg siRNA served as a negative control for LacZ activity and cell viability (i.e., significant reduction in LacZ activity compared to non-transfected cells was attributed to decreased cell viability). HYPERFECT™ (HPF) served herein as a positive transfection control. LacZ activity was determined by measured absorbance at 405 nM with a μQuant plate reader (Bio-Trek Instrument). Refer to Example 1 of the instant application for a description of the LacZ assay.

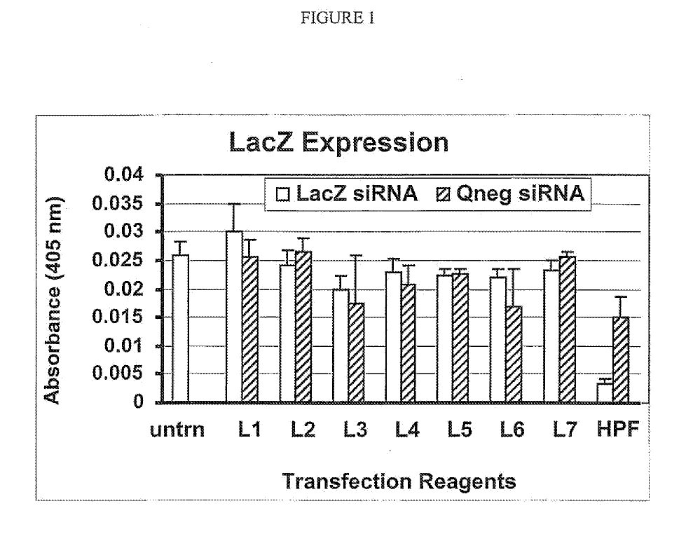

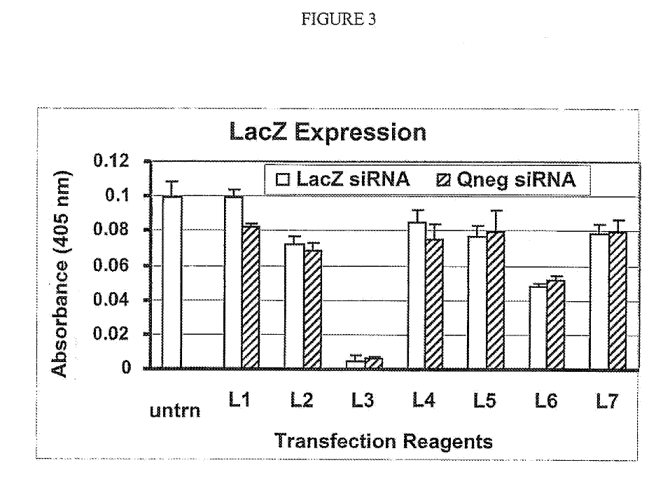

Transfection of 9L/LacZ cells with LacZ siRNA and one or more non-cationic lipids fails to knockdown β-galactosidase activity relative to the Qneg siRNA. The results are shown in FIGS. 1, 2 and 3 for the lipid groups L1 through L7 transfected at a concentration of 7 nM, 20 nM and 60 nM, respectively. The light colored bars in FIGS. 1, 2 and 3 represent the results of transfection with LacZ siRNA while the dark colored bars represent the results of transfection with the Qneg siRNA. The effect of the LacZ siRNA and Qneg siRNA on LacZ activity for each transfection condition was compared.

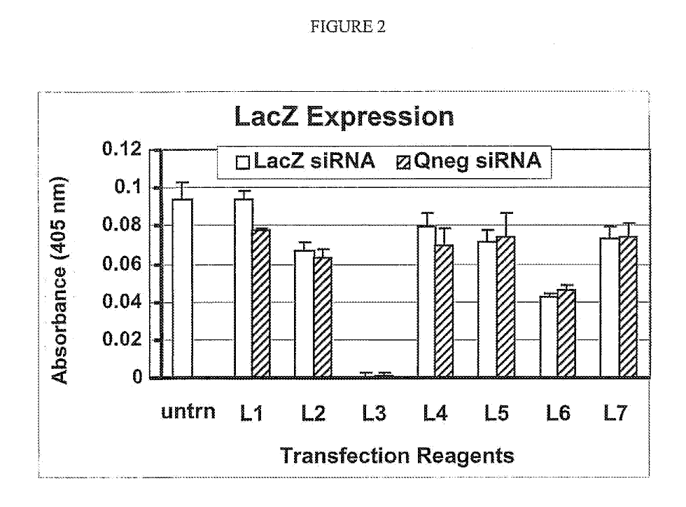

FIGS. 1, 2 and 3 show the LacZ activity of 9L/LacZ cells as measured by absorbance for non-transfected cells and lipid groups L1 through L7 transfected cells. FIG. 2 also includes cells transfected with HPF. The non-transfected (untrn) cells were used as a base-line for comparison with the LacZ and Qneg siRNA transfections. The arrow on the bar graph indicates a significant reduction in LacZ activity (i.e., significant knockdown compared to non-transfected cells). As expected, LacZ activity was present with the non-transfected cells. LacZ activity was reduced (“knockdown”) for cells transfected with the positive transfection control HPF and LacZ siRNA compared to non-transfected (see arrow). At all concentrations with the lipid groups L1 through L7, no significant difference in LacZ activity was observed between 9L/LacZ cells transfected with the LacZ siRNA and the Qneg siRNA indicating that one more neutral lipids is not sufficient to transfect siRNA and permit siRNA mediated degradation of a target RNA.

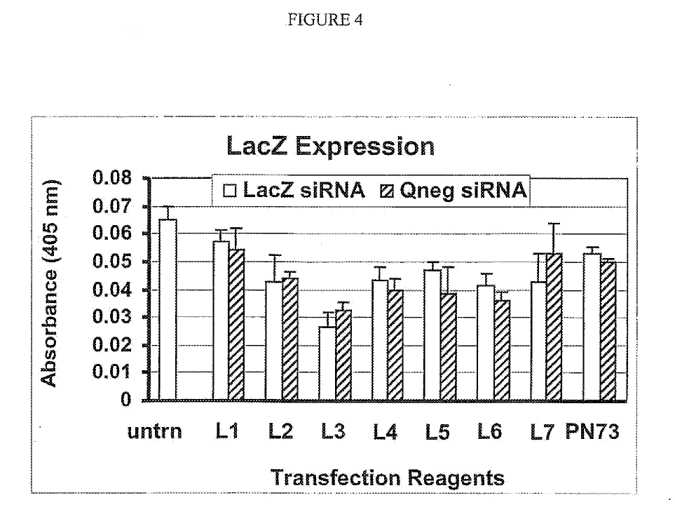

Transfection of 9L/LacZ cells with LacZ siRNA and the combination of one or more non-cationic lipids and the polypeptide PN73 fails to knockdown β-galactosidase activity. The results are shown in FIGS. 4, 5 and 6 for the lipid groups L1 through L7 transfected at a concentration of 7 nM, 20 nM and 60 nM, respectively, in combination with 2 μM of the polypeptide PN73. Again, the light colored bars in FIGS. 4, 5 and 6 represent the results of transfection with LacZ siRNA while the dark colored bars represent the results of transfection with the Qneg siRNA. The effect of the LacZ siRNA and Qneg siRNA on LacZ activity for each transfection condition was compared.

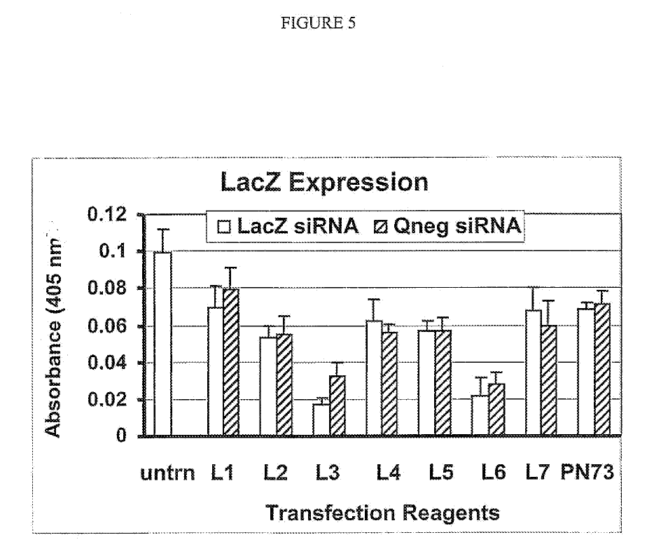

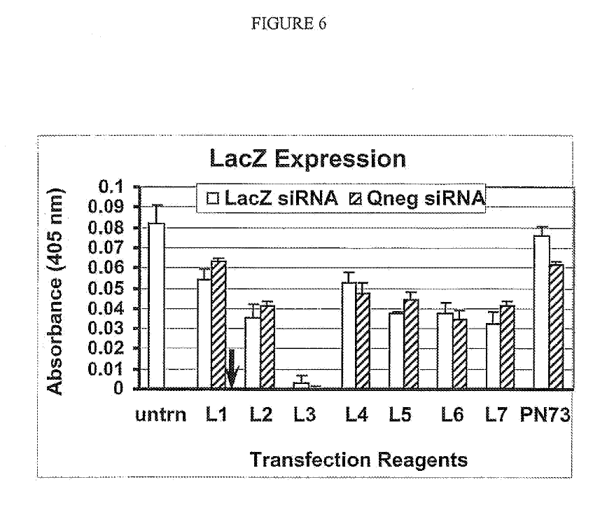

FIGS. 4, 5 and 6 show the LacZ activity of 9L/LacZ cells as measured by absorbance for non-transfected cells, PN73 alone transfected cells and cells transfected with lipid groups L1 through L7 in combination with PN73. The non-transfected (untrn) cells were used as a base-line for comparison with the LacZ and Qneg siRNA transfections. The arrow on the bar graph indicates a significant reduction in LacZ activity (i.e., significant knockdown compared to non-transfected cells). In all instances, transfection of the LacZ siRNA with the polypeptide PN73 in 9L/LacZ cells did not knockdown LacZ activity. As expected, LacZ activity was measured with the non-transfected cells. The transfection performed with lipid groups L1 through L7 at 7 nM and LacZ siRNA failed to knockdown LacZ activity in 9L/LacZ cells (FIG. 4). The transfection performed with the lipid groups L1, L2, L4, L5 and L6 at 20 nM in combination with the polypeptide PN73 and the LacZ siRNA failed to significantly knockdown LacZ activity in 9L/LacZ cells (FIG. 5) compared to the transfection with the Qneg siRNA, indicating that one or more non-cationic lipids combined with the polypeptide PN73 is not sufficient to transfect siRNA and permit siRNA mediated degradation of a target RNA. The transfection performed with the lipid groups L3 and L6 at 20 nM in combination with the polypeptide PN73 and the LacZ siRNA reduced LacZ activity (see arrows); however, LacZ activity was also reduced with the Qneg siRNA indicating that, in general, cell viability was impacted negatively and the reduced LacZ activity was a non-specific result. The transfection performed with the lipid groups L1, L2, L4 through L7 at 60 nM in combination with the polypeptide PN73 and the LacZ siRNA failed to significantly knockdown LacZ activity in 9L/LacZ cells (FIG. 6) compared to the transfection with the Qneg siRNA, indicating that one or more non-cationic lipids in combination with the polypeptide PN73 is not sufficient to transfect siRNA and permit siRNA mediated degradation of a target RNA. The transfection performed with the lipid group L3 at 60 nM in combination with the polypeptide PN73 and the LacZ siRNA reduced LacZ activity (see arrows); however, LacZ activity was also reduced with the Qneg siRNA indicating that, in general, cell viability was impacted negatively.

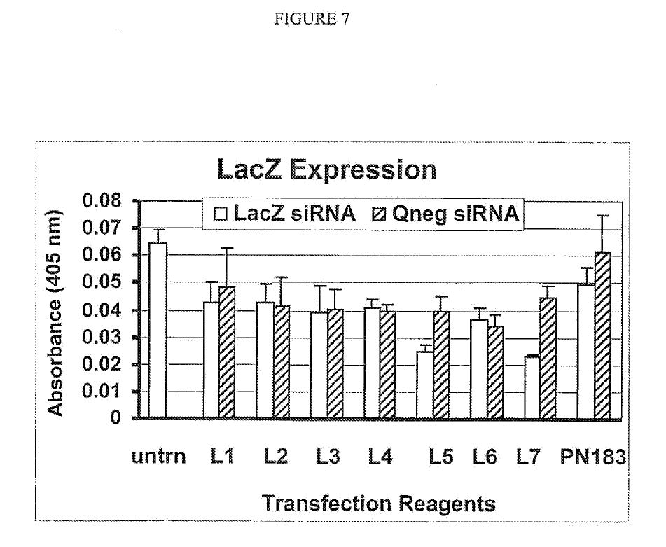

In contrast to the results discussed above, transfection of 9L/LacZ cells with LacZ siRNA and the combination of one or more non-cationic lipids and the exemplary polypeptide PN183 effectively knocks down β-galactosidase activity. The results are shown in FIGS. 7, 8 and 9 for the lipid groups L1 through L7 transfected at a concentration of 7 nM, 20 nM and 60 nM, respectively, in combination with 7 μM of the exemplary polypeptide PN183. Again, the light colored bars in FIGS. 7, 8 and 9 represent the results of transfection with LacZ siRNA while the dark colored bars represent the results of transfection with the Qneg siRNA. The effect of the LacZ siRNA and Qneg siRNA on LacZ activity for each transfection condition was compared.

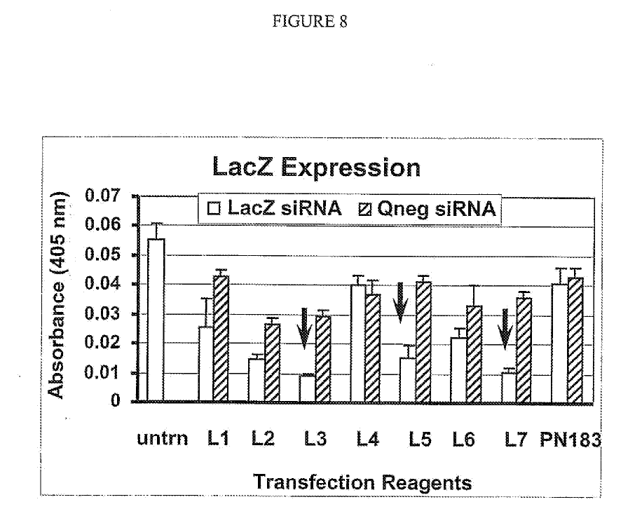

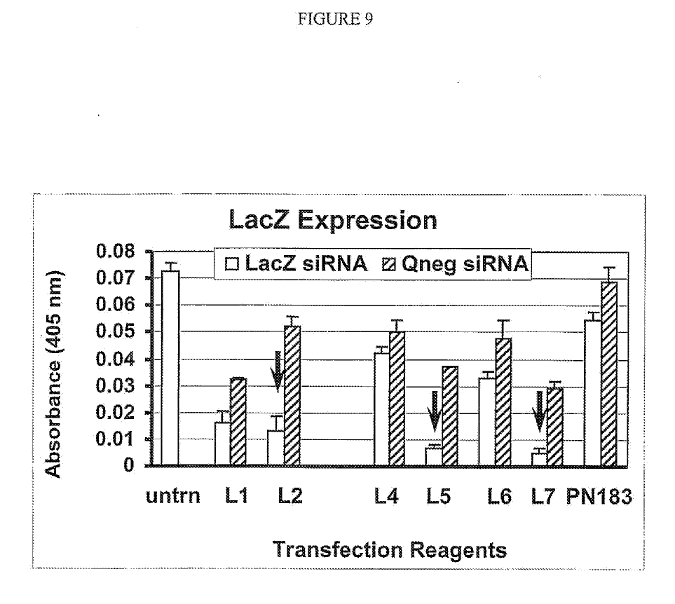

FIGS. 7, 8 and 9 show the LacZ activity of 9L/LacZ cells as measured by absorbance for non-transfected cells, PN183 alone transfected cells and cells transfected with the lipid groups L1 through L7 in combination with PN183. The non-transfected (untrn) cells were used as a base-line for comparison with the LacZ and Qneg siRNA transfections. The arrow on the bar graph indicates a significant reduction in LacZ activity (i.e., significant knockdown compared to non-transfected cells). In all instances, transfection of the LacZ siRNA with the polypeptide PN183 alone in 9L/LacZ cells did not knockdown LacZ activity. As expected, LacZ activity was measured with the non-transfected cells. The transfection performed with lipid groups L5 and L7 at 7 nM in combination with the polypeptide PN183 and the LacZ siRNA reduced LacZ activity in 9L/LacZ (FIG. 7). The transfection performed with the lipid groups L3, L5 and L7 at 20 nM in combination with the polypeptide PN183 and the LacZ siRNA significantly reduced LacZ activity in 9L/LacZ cells (FIG. 8) compared to the transfection with the Qneg siRNA, indicating that one or more non-cationic lipids in combination with the polypeptide PN183 is sufficient to transfect siRNA and permit siRNA mediated degradation of a target RNA. The transfection performed with the lipid groups L1, L2, L4, L5 and L7 at 60 nM in combination with the polypeptide PN183 and the LacZ siRNA significantly reduced LacZ activity in 9L/LacZ cells (FIG. 9) compared to the transfection with the Qneg siRNA, indicating that one or more non-cationic lipids in combination with the polypeptide PN183 is sufficient to transfect siRNA and permit siRNA mediated degradation of a target RNA.

Cells were visualized 72 hours post-transfection for cytotoxic effects by light microscopy at 200× magnification. Cytotoxicity was determined by comparing cell number, cell morphology and cell adhesion within the field of view between non-transfected and transfected cells. The visual-microscopy based assay indicated that cells transfected with non-cationic lipids did not exhibit signs of cytotoxicity (i.e., decreased cell number, abnormal cell morphology or non-adherence) at the concentrations tested.

These data show the surprising and unexpected discovery that siRNA transfected with one or more non-cationic lipids and polypeptide combination effectively reduces the activity of the gene product of the transcript targeted for degradation by the siRNA without a significant negative impact on cell viability. These results further indicate that the combination of one or more non-cationic lipids in combination with a polypeptide overcomes any suppressive effect secondary to delivery of the siRNA with either a lipid or polypeptide alone.

### Example 5

### Effective In Vitro Knockdown of β-Galactosidase Activity by a LacZ siRNA Transfected with a Polypeptide, Non-Cationic Lipid and Cationic Lipid Delivery Vehicle

The present example demonstrates that the addition of an exemplary polypeptide to a non-cationic lipid and cationic lipid transfection mixture (lipid transfection mixture) overcomes the failure of the lipid transfection mixture to permit the effective reduction in activity of the gene product of the transcript targeted for degradation by a transfected siRNA. Further, the addition of the exemplary polypeptide to the lipid transfecture mixture had little to no effect on cytotoxicity and in some cases reduced the level of cytotoxicity observed with the lipid transfecture mixture alone.

In the instant example, transfection efficiency (“uptake”), LacZ knockdown and cytotoxicity were compared for 9L/LacZ cells transfected with a lipid transfecture mixture and 9L/LacZ cells transfected with a combination of a lipid transfecture mixture and an exemplary polypeptide. The lipid transfecture mixtures used in the instant example are illustrated below in Table 5. Each lipid transfecture mixture contained a non-cationic lipid and a cationic lipid at a ration of 1:1, 2:1 and 3:1, respectively, by weight. The final concentration of each lipid transfecture mixture was 3 mg/mL in a final volume of 200 μl. Each lipid listed in Table 5 was stored at a stock concentration of 20 mg/mL. The cationic lipid ANIMAT™ (Ethyl-Nα-dodecanoyl-L-arginate HCL) is a derivative of L-arginine and lauric acid.

The polypeptides PN73 and PN183 were used in the instant example. PN73 was used at a concentration of 2 μM and PN183 was used at 7 M. The LacZ siRNA and the Qneg siRNA were individually transfected with each transfection condition. The Qneg siRNA transfections served base-line control for LacZ activity and cytotoxicity observed with transfections performed with the LacZ siRNA. HYPERFECT™ (HPF) served as a positive transfection control. LacZ activity was determined by measured absorbance at 405 nM with a μQuant plate reader (Bio-Trek Instrument). Refer to Example 1 of the instant application for a description of the LacZ assay used herein.

TABLE 5
Description of Neutral Lipid and Cationic Lipid Transfection Mixtures

| Lipid | Non- | | Non- |
|---|---|---|---|
| Formulation | Cationic | Cationic | Cationic:Cationic |
| ID No. | Lipid | Lipid | Ratio (w/w) |
| 101 | DOPE | DOTAP | 1:1 |
| 102 | DOPE | DOTAP | 2:1 |
| 103 | DOPE | DOTAP | 3:1 |
| 105 | DOPE | DC-Chol | 1:1 |
| 106 | DOPE | DC-Chol | 2:1 |
| 107 | DOPE | DC-Chol | 3:1 |
| 109 | DOPE | 18:1 EPC | 1:1 |
| 110 | DOPE | 18:1 EPC | 2:1 |
| 111 | DOPE | 18:1 EPC | 3:1 |
| 113 | DOPE | 18:0 EPC | 1:1 |
| 114 | DOPE | 18:0 EPC | 2:1 |
| 115 | DOPE | 18:0 EPC | 3:1 |
| 117 | DOPE | 16:0 EPC | 1:1 |
| 118 | DOPE | 16:0 EPC | 2:1 |
| 119 | DOPE | 16:0 EPC | 3:1 |
| 121 | DOPE | AMINAT ™ | 1:1 |
| 122 | DOPE | AMINAT ™ | 2:1 |
| 123 | DOPE | AMINAT ™ | 3:1 |
| 125 | DOPE | 18:0 DDAB | 1:1 |
| 126 | DOPE | 18:0 DDAB | 2:1 |
| 127 | DOPE | 18:0 DDAB | 3:1 |
| 129 | DOPE | 14:0 TAP | 1:1 |
| 130 | DOPE | 14:0 TAP | 2:1 |
| 131 | DOPE | 14:0 TAP | 3:1 |
| 133 | DPhPE | DOTAP | 1:1 |
| 134 | DPhPE | DOTAP | 2:1 |
| 135 | DPhPE | DOTAP | 3:1 |
| 137 | DPhPE | DC-Chol | 1:1 |
| 138 | DPhPE | DC-Chol | 2:1 |
| 139 | DPhPE | DC-Chol | 3:1 |
| 141 | DPhPE | 18:1 EPC | 1:1 |
| 142 | DPhPE | 18:1 EPC | 2:1 |
| 143 | DPhPE | 18:1 EPC | 3:1 |
| 145 | DPhPE | 18:0 EPC | 1:1 |
| 146 | DPhPE | 18:0 EPC | 2:1 |
| 147 | DPhPE | 18:0 EPC | 3:1 |
| 149 | DPhPE | 16:0 EPC | 1:1 |
| 150 | DPhPE | 16:0 EPC | 2:1 |
| 151 | DPhPE | 16:0 EPC | 3:1 |
| 153 | DPhPE | AMINAT ™ | 1:1 |
| 154 | DPhPE | AMINAT ™ | 2:1 |
| 155 | DPhPE | AMINAT ™ | 3:1 |
| 157 | DPhPE | 18:0 DDAB | 1:1 |
| 158 | DPhPE | 18:0 DDAB | 2:1 |
| 159 | DPhPE | 18:0 DDAB | 3:1 |
| 161 | DPhPE | 14:0 TAP | 1:1 |
| 162 | DPhPE | 14:0 TAP | 2:1 |
| 163 | DPhPE | 14:0 TAP | 3:1 |
| 165 | CHEMS | DOTAP | 1:1 |
| 166 | CHEMS | DOTAP | 2:1 |
| 167 | CHEMS | DOTAP | 3:1 |
| 169 | CHEMS | DC-Chol | 1:1 |
| 170 | CHEMS | DC-Chol | 2:1 |
| 171 | CHEMS | DC-Chol | 3:1 |
| 173 | CHEMS | 18:1 EPC | 1:1 |
| 174 | CHEMS | 18:1 EPC | 2:1 |
| 175 | CHEMS | 18:1 EPC | 3:1 |
| 177 | CHEMS | 18:0 EPC | 1:1 |
| 178 | CHEMS | 18:0 EPC | 2:1 |
| 179 | CHEMS | 18:0 EPC | 3:1 |
| 181 | CHEMS | 16:0 EPC | 1:1 |
| 182 | CHEMS | 16:0 EPC | 2:1 |
| 183 | CHEMS | 16:0 EPC | 3:1 |
| 185 | CHEMS | AMINAT ™ | 1:1 |
| 186 | CHEMS | AMINAT ™ | 2:1 |
| 187 | CHEMS | AMINAT ™ | 3:1 |
| 189 | CHEMS | 18:0 DDAB | 1:1 |
| 190 | CHEMS | 18:0 DDAB | 2:1 |
| 191 | CHEMS | 18:0 DDAB | 3:1 |
| 193 | CHEMS | 14:0 TAP | 1:1 |
| 194 | CHEMS | 14:0 TAP | 2:1 |
| 195 | CHEMS | 14:0 TAP | 3:1 |
| 197 | Chol | DOTAP | 1:1 |
| 198 | Chol | DOTAP | 2:1 |
| 199 | Chol | DOTAP | 3:1 |
| 201 | Chol | DC-Chol | 1:1 |
| 202 | Chol | DC-Chol | 2:1 |
| 203 | Chol | DC-Chol | 3:1 |
| 205 | Chol | 18:1 EPC | 1:1 |
| 206 | Chol | 18:1 EPC | 2:1 |
| 207 | Chol | 18:1 EPC | 3:1 |
| 209 | Chol | 18:0 EPC | 1:1 |
| 210 | Chol | 18:0 EPC | 2:1 |
| 211 | Chol | 18:0 EPC | 3:1 |
| 213 | Chol | 16:0 EPC | 1:1 |
| 214 | Chol | 16:0 EPC | 2:1 |
| 215 | Chol | 16:0 EPC | 3:1 |
| 217 | Chol | AMINAT ™ | 1:1 |
| 218 | Chol | AMINAT ™ | 2:1 |
| 219 | Chol | AMINAT ™ | 3:1 |
| 221 | Chol | 18:0 DDAB | 1:1 |
| 222 | Chol | 18:0 DDAB | 2:1 |
| 223 | Chol | 18:0 DDAB | 3:1 |
| 225 | Chol | 14:0 TAP | 1:1 |
| 226 | Chol | 14:0 TAP | 2:1 |
| 227 | Chol | 14:0 TAP | 3:1 |
| 229 | DSPC | DOTAP | 1:1 |
| 230 | DSPC | DOTAP | 2:1 |
| 231 | DSPC | DOTAP | 3:1 |
| 233 | DSPC | DC-Chol | 1:1 |
| 234 | DSPC | DC-Chol | 2:1 |
| 235 | DSPC | DC-Chol | 3:1 |
| 237 | DSPC | 18:1 EPC | 1:1 |
| 238 | DSPC | 18:1 EPC | 2:1 |
| 239 | DSPC | 18:1 EPC | 3:1 |
| 241 | DSPC | 18:0 EPC | 1:1 |
| 242 | DSPC | 18:0 EPC | 2:1 |
| 243 | DSPC | 18:0 EPC | 3:1 |
| 245 | DSPC | 16:0 EPC | 1:1 |
| 246 | DSPC | 16:0 EPC | 2:1 |
| 247 | DSPC | 16:0 EPC | 3:1 |
| 249 | DSPC | AMINAT ™ | 1:1 |
| 250 | DSPC | AMINAT ™ | 2:1 |
| 251 | DSPC | AMINAT ™ | 3:1 |
| 253 | DSPC | 18:0 DDAB | 1:1 |
| 254 | DSPC | 18:0 DDAB | 2:1 |
| 255 | DSPC | 18:0 DDAB | 3:1 |
| 257 | DSPC | 14:0 TAP | 1:1 |
| 258 | DSPC | 14:0 TAP | 2:1 |
| 259 | DSPC | 14:0 TAP | 3:1 |
| 261 | DPPC | DOTAP | 1:1 |
| 262 | DPPC | DOTAP | 2:1 |
| 263 | DPPC | DOTAP | 3:1 |
| 265 | DPPC | DC-Chol | 1:1 |
| 266 | DPPC | DC-Chol | 2:1 |
| 267 | DPPC | DC-Chol | 3:1 |
| 269 | DPPC | 18:1 EPC | 1:1 |
| 270 | DPPC | 18:1 EPC | 2:1 |
| 271 | DPPC | 18:1 EPC | 3:1 |
| 273 | DPPC | 18:0 EPC | 1:1 |
| 274 | DPPC | 18:0 EPC | 2:1 |
| 275 | DPPC | 18:0 EPC | 3:1 |
| 277 | DPPC | 16:0 EPC | 1:1 |
| 278 | DPPC | 16:0 EPC | 2:1 |
| 279 | DPPC | 16:0 EPC | 3:1 |
| 281 | DPPC | AMINAT ™ | 1:1 |
| 282 | DPPC | AMINAT ™ | 2:1 |
| 283 | DPPC | AMINAT ™ | 3:1 |
| 285 | DPPC | 18:0 DDAB | 1:1 |
| 286 | DPPC | 18:0 DDAB | 2:1 |
| 287 | DPPC | 18:0 DDAB | 3:1 |
| 289 | DPPC | 14:0 TAP | 1:1 |
| 290 | DPPC | 14:0 TAP | 2:1 |
| 291 | DPPC | 14:0 TAP | 3:1 |
| 293 | DDPC | DOTAP | 1:1 |
| 294 | DDPC | DOTAP | 2:1 |
| 295 | DDPC | DOTAP | 3:1 |
| 297 | DDPC | DC-Chol | 1:1 |
| 298 | DDPC | DC-Chol | 2:1 |
| 299 | DDPC | DC-Chol | 3:1 |
| 301 | DDPC | 18:1 EPC | 1:1 |
| 302 | DDPC | 18:1 EPC | 2:1 |
| 303 | DDPC | 18:1 EPC | 3:1 |
| 305 | DDPC | 18:0 EPC | 1:1 |
| 306 | DDPC | 18:0 EPC | 2:1 |
| 307 | DDPC | 18:0 EPC | 3:1 |
| 309 | DDPC | 16:0 EPC | 1:1 |
| 310 | DDPC | 16:0 EPC | 2:1 |
| 311 | DDPC | 16:0 EPC | 3:1 |
| 313 | DDPC | AMINAT ™ | 1:1 |
| 314 | DDPC | AMINAT ™ | 2:1 |
| 315 | DDPC | AMINAT ™ | 3:1 |
| 317 | DDPC | 18:0 DDAB | 1:1 |
| 318 | DDPC | 18:0 DDAB | 2:1 |
| 319 | DDPC | 18:0 DDAB | 3:1 |
| 321 | DDPC | 14:0 TAP | 1:1 |
| 322 | DDPC | 14:0 TAP | 2:1 |
| 323 | DDPC | 14:0 TAP | 3:1 |

Table 6 below summarizes the transfection efficiency (“uptake”), LacZ knockdown and cytotoxicity for 9L/LacZ cells transfected with the LacZ siRNA and the lipid transfecture mixtures listed in Table 6 in the presence or absence of either the polypeptide PN73 or PN183 relative to 9L/LacZ cells transfected with the Qneg siRNA with the same transfection conditions and after normalization to protein levels. Cytotoxicity was determined by analyzing the LacZ protein concentrations of cells transfected with the Qneg siRNA. The Qneg siRNA does not target the LacZ transcript and, thus, a reduction in LacZ activity relative to non-transfected cells was attributed to reduced cell proliferation resulting from Qneg siRNA transfection induced cytotoxicity. Therefore, the concentration of the protein is used as the indication of toxicity of transfection reagents.

The LacZ siRNA transfection efficiency (uptake) results for the lipid transfection mixture in the presence and absence of either the polypeptide PN73 or PN183 are shown in Table 6 categorized on a relative scale by the presence of a “−” indicating no siRNA uptake was observed; “+/−” indicating limited detectable levels of siRNA uptake were observed; “+” indicating fair siRNA uptake was observed; “++” indicating good siRNA uptake was observed and “+++” and “++++” indicating an excellent level of siRNA uptake was observed. Thus, in general, more +'s indicate greater transfection efficiency.

The LacZ activity knockdown (KD) results for the lipid transfection mixture in the presence and absence of either the exemplary polypeptide PN73 or PN183 are also shown in Table 6 and categorized with the following scale: “−” indicates 0% to 30% KD; “+” indicates 30 to 40% KD; “++” indicates 40 to 60% KD; “+++” indicates 60 to 80% KD and “++++” indicates 80% or greater KD. Thus, in general, more +'s indicate greater knockdown activity.

The cytotoxicity results for the lipid transfection mixture in the presence and absence of either the exemplary polypeptide PN73 or PN183 are also shown in Table 6 and categorized with the following scale: “−” indicates that protein levels of 80% or greater compared to non-transfected 9L/LacZ cells was present; “+” indicates that protein levels of 60 to 80% compared to non-transfected 9L/LacZ cells was present; “++” indicates that protein levels of 40 to 60% compared to non-transfected 9L/LacZ cells was present; “+++” indicates that protein levels of 20 to 40% compared to non-transfected 9L/LacZ cells was present and “++++” indicates that protein levels of less than 20% compared to non-transfected 9L/LacZ cells was present. Thus, in general, a smaller number of +'s indicates lower cytotoxicity.

TABLE 6
Comparative Summary of siRNA Uptake, LacZ Activity Knockdown and
Cytotoxicity of 9L/LacZ Cells Transfected with a Lipid Transfecture Mixture
in the Presence or Absence of an Exemplary Polypeptides PN73 or PN183.

| | Lipid Transfecture | | | Lipid Transfecture | | | Lipid Transfecture | | |
|---|---|---|---|---|---|---|---|---|---|
| Lipid | Mixture | | | Mixture (10 nM) + | | | Mixture (10 nM) + | | |
| Formulation | (10 nM) | | | PN183 (7 μM) | | | PN73 (2 μM) | | |
| ID No. | Uptake | KD | Toxicity | Uptake | KD | Toxicity | Uptake | KD | Toxicity |
| Non- | N/A | N/A | N/A | N/A | N/A | N/A | N/A | N/A | N/A |
| transfected | | | | | | | | | |
| HiPerFect | +++ | ++++ | +++ | +++ | ++++ | ++ | +++ | ++++ | ++ |
| Peptide alone | N/A | N/A | N/A | ++ | − | − | +++ | − | − |
| 101 | ++++ | +++ | ++ | ++++ | +++ | + | ++++ | +++ | + |
| 102 | ++++ | +++ | + | ++++ | +++ | + | ++++ | +++ | + |
| 103 | ++++ | ++++ | +++ | ++++ | ++ | ++ | ++++ | +++ | ++ |
| 105 | ++++ | +++ | +++ | +++ | ++ | +++ | ++++ | +++ | +++ |
| 106 | ++++ | +++ | ++ | ++++ | ++ | +++ | ++++ | +++ | +++ |
| 107 | ++++ | ++++ | ++++ | +++ | +++ | ++++ | ++++ | +++ | ++++ |
| 121 | +/− | − | − | ++ | ++ | − | +++ | − | − |
| 122 | − | − | − | ++ | ++ | − | +++ | − | − |
| 123 | +/− | − | − | ++ | ++ | − | +++ | − | − |
| 129 | +++ | ++ | + | ++++ | ++ | − | ++++ | ++ | − |
| 130 | +++ | +++ | + | ++++ | +++ | − | ++++ | ++ | − |
| 131 | +++ | +++ | + | ++++ | ++ | + | ++++ | ++ | + |
| 133 | +++ | +++ | + | ++++ | +++ | ++ | +++ | ++++ | ++ |
| 134 | ++ | ++ | − | ++++ | +++ | + | +++ | ++ | + |
| 135 | ++ | + | − | ++++ | ++ | + | +++ | − | + |
| 137 | +++ | ++ | − | ++++ | − | ++ | ++++ | − | ++ |
| 138 | +++ | − | − | ++++ | − | ++ | ++++ | − | ++ |
| 139 | ++ | − | − | ++++ | − | ++ | +++ | − | ++ |
| 153 | − | − | − | ++ | − | − | +++ | − | − |
| 154 | − | − | − | ++ | − | − | +++ | − | − |
| 155 | − | − | − | ++ | − | − | +++ | − | − |
| 161 | + | − | − | ++ | − | − | +++ | − | − |
| 162 | + | − | − | ++ | − | − | +++ | − | − |
| 163 | + | − | − | ++ | − | − | ++ | − | − |
| 165 | + | ++ | + | ++ | + | − | +++ | − | − |
| 166 | − | − | − | ++ | − | − | +++ | − | − |
| 167 | − | − | + | ++ | − | − | +++ | − | − |
| 169 | − | − | ++ | ++ | − | − | +++ | − | − |
| 170 | − | − | + | ++ | − | − | +++ | − | − |
| 171 | − | − | + | ++ | − | + | +++ | − | + |
| 185 | − | − | + | ++ | − | − | +++ | − | − |
| 186 | − | − | + | ++ | − | − | +++ | − | − |
| 187 | − | − | ++ | ++ | − | + | ++++ | − | + |
| 193 | − | − | − | ++ | − | − | +++ | − | − |
| 194 | − | − | − | ++ | − | + | +++ | − | + |
| 195 | − | − | − | +++ | − | + | ++++ | − | + |
| 197 | ++++ | +++ | ++++ | ++++ | ++++ | +++ | +++ | +++ | ++++ |
| 198 | ++++ | +++ | ++ | ++++ | ++++ | +++ | +++ | +++ | +++ |
| 199 | ++++ | ++ | + | ++++ | +++ | ++ | +++ | +++ | ++ |
| 201 | ++++ | ++ | +++ | ++++ | +++ | +++ | ++++ | +++ | ++++ |
| 202 | +/− | − | − | ++ | − | − | +++ | − | − |
| 203 | ++ | − | − | ++ | − | − | +++ | − | − |
| 217 | +/− | − | + | ++ | − | + | N/D | N/D | N/D |
| 218 | − | − | + | ++ | − | − | N/D | N/D | N/D |
| 219 | − | − | + | ++ | − | + | N/D | N/D | N/D |
| 225 | +++ | − | + | ++ | − | + | N/D | N/D | N/D |
| 226 | +++ | − | + | ++ | − | − | N/D | N/D | N/D |
| 227 | +++ | − | + | ++ | − | − | N/D | N/D | N/D |
| 229 | +++ | − | + | +++ | − | − | N/D | N/D | N/D |
| 230 | ++ | − | − | +++ | − | − | N/D | N/D | N/D |
| 231 | ++ | − | − | ++ | − | − | N/D | N/D | N/D |
| 233 | +++ | ++ | ++ | +++ | ++ | ++ | N/D | N/D | N/D |
| 234 | +++ | − | − | +++ | − | − | N/D | N/D | N/D |
| 235 | +++ | + | + | +++ | − | − | N/D | N/D | N/D |
| 249 | − | − | + | ++ | − | + | N/D | N/D | N/D |
| 250 | − | − | + | ++ | − | + | N/D | N/D | N/D |
| 251 | − | − | ++ | ++ | − | + | N/D | N/D | N/D |
| 257 | ++ | − | + | ++ | − | − | N/D | N/D | N/D |
| 258 | + | − | + | ++ | − | − | N/D | N/D | N/D |
| 259 | ++ | − | − | ++ | − | − | N/D | N/D | N/D |
| 261 | +++ | ++ | − | +++ | − | − | N/D | N/D | N/D |
| 262 | +++ | − | − | +++ | − | − | N/D | N/D | N/D |
| 263 | ++ | − | − | +++ | − | − | N/D | N/D | N/D |
| 265 | ++ | + | − | ++++ | − | − | N/D | N/D | N/D |
| 266 | ++ | + | − | ++++ | − | + | N/D | N/D | N/D |
| 267 | ++ | + | − | ++++ | − | − | N/D | N/D | N/D |
| 281 | +/− | − | − | ++ | − | − | N/D | N/D | N/D |
| 282 | − | − | − | + | − | − | N/D | N/D | N/D |
| 283 | − | − | − | + | − | − | N/D | N/D | N/D |
| 289 | + | − | + | ++ | − | − | N/D | N/D | N/D |
| 290 | + | − | − | ++ | − | − | N/D | N/D | N/D |
| 291 | + | − | − | ++ | − | − | N/D | N/D | N/D |

The results in Table 6 show the surprising and unexpected discovery that the addition of an exemplary polypeptide to a non-cationic lipid and cationic lipid transfection mixture (lipid transfection mixture) overcomes the failure of the lipid transfection mixture to permit the effective reduction in activity of the gene product of the transcript targeted for degradation by a transfected siRNA (e.g., Lipid Formulation ID Nos. 121, 122 and 123). Further, the addition of the exemplary polypeptide to the lipid transfecture mixture did not induce cytotoxicity. Further, in some cases, the addition of the exemplary polypeptide to the lipid transfecture mixture reduced the level of cytotoxicity observed with the lipid transfecture mixture alone.

### Example 6

### Chemical Structures of Exemplary Lipids of the Present Invention

The present example illustrates the chemical structure of exemplary lipids of the present invention including non-cationic and cationic lipids. The chemical structures are solely for purposes of illustration, and are not intended to limit the scope of the invention.

The chemical structure of 1,2-Dipalmitoyl-sn-Glycero-3-[Phospho-rac-(1-glycerol)] (sodium salt; DPPG) is as follows:

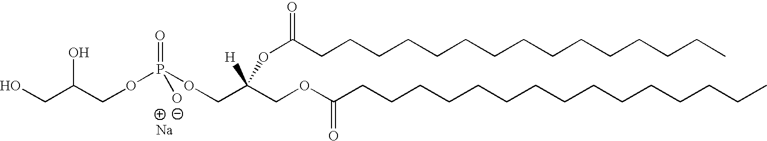

The chemical structure of 1,2-Dipalmitoyl-sn-Glycero-3-Phosphocholine (DPPC) is as follows:

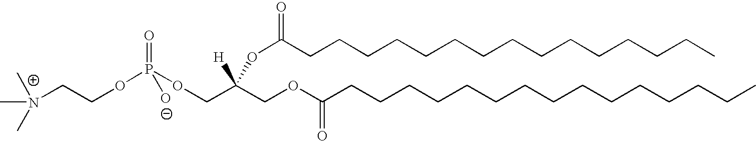

The chemical structure of 1,2-Distearoyl-sn-Glycero-3-Phosphocholine (DSPC) is as follows:

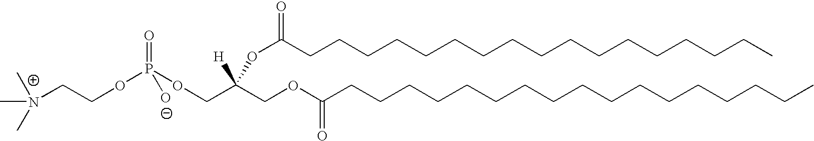

The chemical structure of 1,2-Distearoyl-sn-Glycero-3-Phosphoethanolamine (DSPE) is as follows:

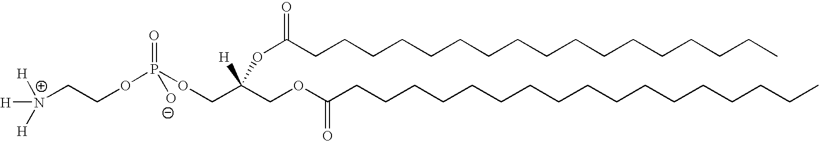

The chemical structure of 1,2-Dimyristoylamido-1,2-Deoxyphosphatidyl Choline (DDPC) is as follows:

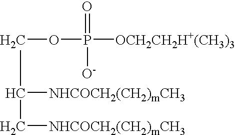

The chemical structure of 1,2-Dioleoyl-sn-Glycero-3-Phosphoethanolamine (DOPE) is as follows:

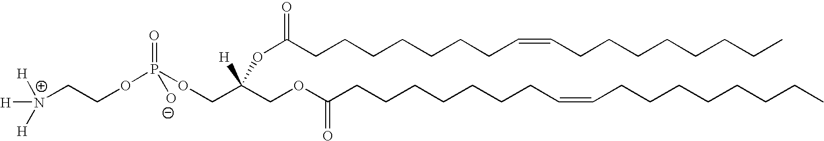

The chemical structure of 1,2-Dioleoyl-sn-Glycero-3-Phosphocholine (DOPC) is as follows:

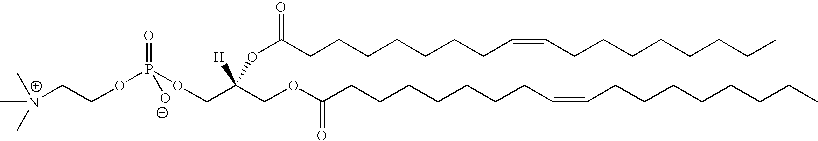

The chemical structure of 3β-[N-(N′,N′-Dimethylaminoethane)-carbamoyl] Cholesterol Hydrochloride (DC-Cholesterol HCl) is as follows:

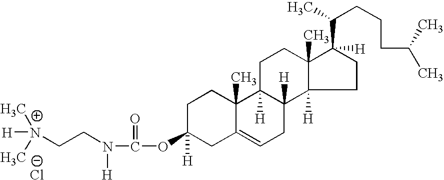

The chemical structure of Cholesterol is as follows:

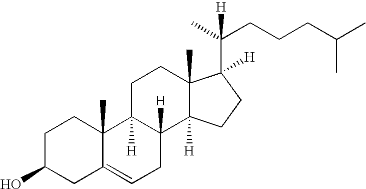

The chemical structure of 1,2-Dioleoyl-3-Trimethylammonium-Propane (chloride salt; DOTAP) is as follows:

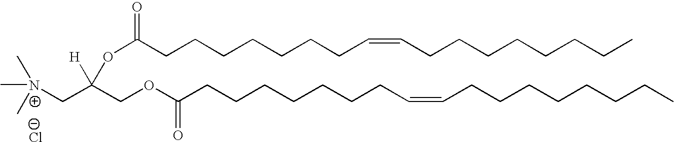

The chemical structure of 1,2-Dioleoyl-sn-Glycero-3-Ethylphosphocholine (chloride salt) is as follows:

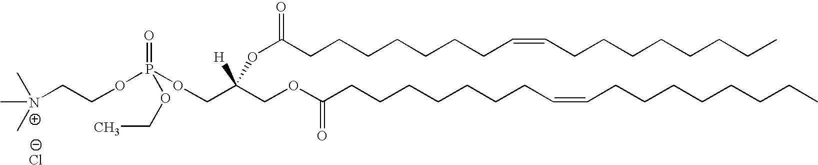

The chemical structure of 1,2-Distearoyl-sn-Glycero-3-Ethylphosphocholine (chloride salt) is as follows:

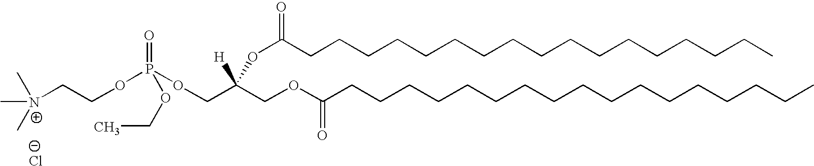

The chemical structure of 1,2-Dipalmitoyl-sn-Glycero-3-Ethylphosphocholine (Chloride Salt) is as follows:

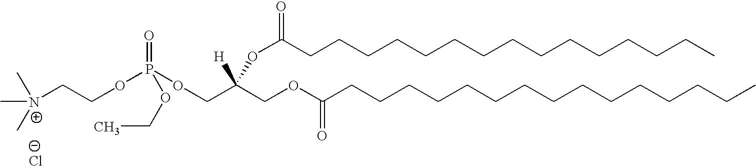

The chemical structure of 1,2-Dimyristoyl-3-Trimethylammonium-Propane (chloride salt) is as follows:

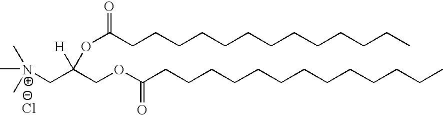

The chemical structure of 1,2-Distearoyl-3-Trimethylammonium-Propane (chloride salt) is as follows:

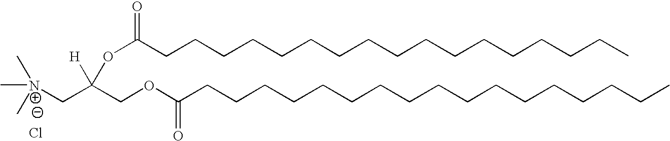

The chemical structure of Dimethyldioctadecylammonium (bromide salt; DDAB) is as follows:

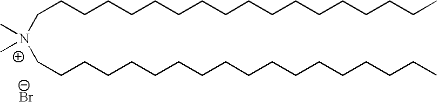

The chemical structure of 1,2-Diphytanoyl-sn-Glycero-3-Phosphocholine (DPhPC) is as follows:

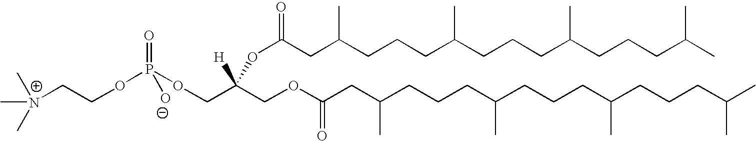

The chemical structure of 1,2-Dimyristoyl-sn-Glycero-3-[Phospho-rac-(1-glycerol)] (sodium salt; DMPG) is as follows:

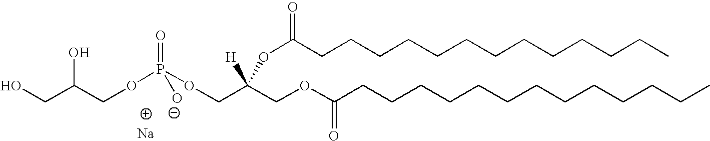

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

What is claimed is:

1. A pharmaceutical composition comprising:
a. a double-stranded RNA (dsRNA) molecule having a double-stranded region of from about 15 to about 40 base pairs;
b. a peptide, comprising a hydrophobic region and a cationic region; and
c. a non-cationic phospholipid.

2. The composition of claim 1, wherein the dsRNA molecule is an siRNA or an shRNA.

3. The composition of claim 1, wherein the dsRNA molecule has a 3′ overhang.

4. The composition of claim 1, wherein the dsRNA molecule has a 3′ overhang containing a deoxythymidine (dT).

5. The composition of claim 1, wherein the non-cationic lipid is selected from the group consisting of a neutral lipid, zwitterionic lipid and an anionic lipid.

6. The composition of claim 1, wherein the non-cationic phospholipid is selected from the group consisting of 1,2-dioleoyl-sn-glycero-3-phosphoethanolamine (DOPE); diphytanoylphosphatidylethanolamine (DPhPE); cholesterol hemisuccinate salt (CHEMS); cholesterol; 1,2-distearoyl-sn-glycero-3-phosphocholine (DSPC); 1,2-dipalmitoyl-sn-glycero-3-phosphocholine (DPPC) and 1,2-dimyristoylamido-1,2-deoxyphosphatidylcholine (DDPC), 1,2-dipalmitoyl-sn-glycero-3-phosphatidylserine (DPPS), 1,2-distearoyl-sn-glycero-3-phosphatidylserine (DSPS), and phosphatidic acid derived from any one of the above lipids.

7. The composition of claim 1, wherein the peptide has at least three cationic amino acids in a five amino acid region within ten amino acids from a terminus of the peptide.

8. The composition of claim 1, wherein the peptide has at least three hydrophobic amino acids in a five amino acid region within ten amino acids from a terminus of the peptide.

9. The composition of claim 1, wherein the hydrophobic amino acids are near to one terminus of the peptide and the cationic amino acids are near the other terminus of the peptide.

10. The composition of claim 1, wherein the hydrophobic amino acids are separated from the cationic amino acids by at least three amino acids.

11. The composition of claim 1 wherein the amino acid sequence of the peptide is:

| | |
|---|---|
| KETWWETWWTEWSQPGRKKRRQRRRPPQ. | (SEQ ID NO: 36) |

12. The composition of claim 1, further comprising a cationic lipid.

13. The composition of claim 12, wherein the ratio of non-cationic to cationic lipid is greater than about 1:1 (w:w).

14. A pharmaceutical composition for inhibiting expression of a target gene in a cell, comprising:
a. a double-stranded RNA (dsRNA) molecule having a double-stranded region of from about 15 to about 40 base pairs, and having sequence homology to a sequence of the gene;
b. a peptide, comprising a hydrophobic region and a cationic region; and
c. a non-cationic phospholipid.

15. The composition of claim 14, wherein the non-cationic lipid is selected from the group consisting of a neutral lipid, zwitterionic lipid and an anionic lipid.

16. The composition of claim 14, wherein the non-cationic phospholipid is selected from the group consisting of 1,2-dioleoyl-sn-glycero-3-phosphoethanolamine (DOPE); diphytanoylphosphatidylethanolamine (DPhPE); cholesterol hemisuccinate salt (CHEMS); cholesterol; 1,2-distearoyl-sn-glycero-3-phosphocholine (DSPC); 1,2-dipalmitoyl-sn-glycero-3-phosphocholine (DPPC) and 1,2-dimyristoylamido-1,2-deoxyphosphatidylcholine (DDPC), 1,2-dipalmitoyl-sn-glycero-3-phosphatidylserine (DPPS), 1,2-distearoyl-sn-glycero-3-phosphatidylserine (DSPS), and phosphatidic acid derived from any one of the above lipids.

17. The composition of claim 14, wherein the peptide has at least three cationic amino acids in a five amino acid region within ten amino acids from a terminus of the peptide.

18. The composition of claim 14, wherein the peptide as at least three hydrophobic amino acids in a five amino acid region within ten amino acids from a terminus of the peptide.

19. The composition of claim 14, wherein the hydrophobic amino acids are near to one terminus of the peptide and the cationic amino acids are near the other terminus of the peptide.

20. The composition of claim 14, wherein the hydrophobic amino acids are separated from the cationic amino acids by at least three amino acids.

21. The composition of claim 14, wherein the amino acid sequence of the peptide is:

| | |
|---|---|
| KETWWETWWTEWSQPGRKKRRQRRRPPQ. | (SEQ ID NO: 36) |

22. The composition of claim 14, further comprising a cationic lipid.

23. The composition of claim 22, wherein the ratio of non-cationic to cationic lipid is greater than about 1:1 (w:w).

24. A method for delivering a RNA molecule to a cell, comprising:
a. preparing a composition comprising:
i. a double-stranded RNA (dsRNA) molecule having a double-stranded of from about 15 to about 40 base pairs;
ii. a peptide, comprising a hydrophobic region and a cationic region; and
iii. a non-cationic phospholipid; and
a. treating a cell with said composition.

25. A method for inhibiting expression of a gene in a cell comprising:
a. preparing a pharmaceutical composition comprising:
i. a double-stranded RNA (dsRNA) molecule having a double-stranded region of from about 15 to about 40 base pairs, and having sequence homology to a sequence of the gene;
ii. a peptide, comprising a hydrophobic region and a cationic region; and
iii. a non-cationic phospholipid; and
b. treating a cell with said pharmaceutical composition.

26. A method for inhibiting expression of a gene in a mammal comprising:
a. preparing a pharmaceutical composition comprising:
i. a double-stranded RNA (dsRNA) molecule having a double-stranded region of from about 15 to about 40 base pairs, and having sequence homology to a sequence of the gene;
ii. a peptide, comprising a hydrophobic region and a cationic region; and
iii. a non-cationic phospholipid; and
b. administering said pharmaceutical composition to said mammal.
